(19) United States
(12) Patent Application Publication
(10) Pub. No.: US 2008/0057061 A1
(43) Pub. Date: Mar. 6, 2008

(54) Method of Treating Diseases and Disorders

(75) Inventor: Donald Harn, Pembroke, MA (US)

Correspondence Address: RONALD I. EISENSTEIN;NIXON PEABODY LLP, 100 SUMMER STREET, BOSTON, MA, 02110, US

(73) Assignee: President and Fellows of Harvard College, Cambridge, MA (US)

(21) Appl. No.: 11/791,360

(22) PCT Filed: Nov. 22, 2005
(86) PCT No.: PCT/US05/42597

§ 371 Date: Nov. 13, 2007

Related U.S. Application Data
(60) Provisional application No. 60/630,172, filed on Nov. 22, 2004.

(51) Int. Cl. A61K 39/395; A61P 31/18

(52) U.S. Cl. 424/135.1; 424/130.1; 424/141.1; 514/789

(57) ABSTRACT

We have discovered that targeting carbohydrates on cell, or viral particle surfaces, such as using antibodies against such carbohydrates also called glycans provide a new tool to treat diseases and disorders, such as viral diseases and malignant tumors. Therefore, the present invention provides methods for treating individuals affected with diseases and disorders, for example viral infections, such as lentiviral infections, and malignant tumors, using molecules that bind carbohydrates that are expressed on the surface of the viral particle or a cell, for example antibodies against Lewis X-antigen.

### CROSS-REFERENCE TO RELATED APPLICATIONS

The present invention claims the benefit under 35 U.S.C. §119 of U.S. provisional application No. 60/630,172, filed Nov. 22, 2004, the content of which is herein incorporated by reference in its entirety.

### BACKGROUND OF THE INVENTION

Antibodies directed against carbohydrate determinants are of fundamental importance in the immunity against bacterial and viral pathogens and in the fight against malignancies.

Many proteins that are expressed, particularly on the surface of cells and on the coat of viral particles, are posttranslationally modified by attaching carbohydrates on them. Accordingly, such carbohydrates can serve as targets for antibody production. Such antibodies would be a useful tool to fight diseases and disorders where carbohydrate-coated proteins are part of the invading agent's composition, whether it be a cell or a viral particle.

Development of useful antibodies has been hindered by difficulties in detection, synthesis and isolation of disease-specific carbohydrates. These carbohydrates may also hinder targeting antibodies that have been raised against synthetic proteins or peptides because they mask the surface protein epitopes.

It would be useful to identify disease-causing agent specific carbohydrates and prepare molecules that can bind them. These would provide useful tools for treatment, and prevention of many diseases.

For example, it is known that peptide and protein antigens used in attempts to invoke immune response against lentiviruses, such as HIV-1 infection have been generally relatively ineffective. One of the reasons for this ineffectiveness is the high viral reverse transcriptase (RT) activity that generates viral escape mutants. In addition to the RT activity, the abundant glycosylation of the viral envelope is suspected to block anti-protein antibody access to their targets. This phenomenon has been referred to as the viral “Glycan Shield.”

Glycoproteins are known to be targets for some antiviral agents. For example, Cyanovirin is an antiviral that binds to glycoprotein 120 (gp 120) present in the surface of HIV-1. Also, an artificial human Monoclonal antibody (hmAb) 2G12 has been described to recognize glycans on gp120 thereby neutralizing the virus. However, neutralizing the primary lentivirus, such as HIV-1 isolates has been very difficult and production of a vaccine targeting HIV-1 envelope glycans has until now, not been possible due to the lack of reagents and knowledge on structures of envelope glycans.

In addition, it is known that a minimally effective response to malignant cells requires target antigens on the tumor cells, effector precursors capable of recognizing them, and regulatory cells that foster the development of appropriate effector responses. Several hypotheses have been advanced to explain why the system often fails. These include poor immunogenicity of tumor associated antigens (TAA), the weak ability of solid tumor cells to deliver costimulatory signals, the action of cytokines and growth factors, such as VEGF, IL-10 and TGF, that suppress effector cell maturation and functional ability, and the ability of regulatory T cells (Treg) to suppress the development of responses against the tumor. However, reports that tumor immunity that develops initially is lost before the tumor is destroyed, suggests that mechanisms exist to suppress tumor immunity even after it develops. The role of carbohydrates in tumor immunology has been under investigation by companies, such as Glycotech (Gaithersburg, Md.), but the results of antibodies against tumor-specific carbohydrates are still in their infancy.

### SUMMARY OF THE INVENTION

We have discovered that targeting carbohydrates on cell, or viral particle surfaces, such as using antibodies against such carbohydrates also called glycans provide a new tool to treat diseases and disorders, such as viral diseases and malignant tumors. Therefore, the present invention provides methods for treating individuals affected with diseases and disorders, for example viral infections, such as lentiviral infections, and malignant tumors, using molecules that bind carbohydrates that are expressed on the surface of the viral particle or a cell, for example antibodies against Lewis X-antigen.

We showed that binding of carbohydrates, by for example antibodies, prevents lentivial infections by using antibodies against carbohydrates. We also found that antibodies against carbohydrates can be used to prevent or delay decrease the host's Th1 protective immune response.

Together, these findings demonstrate that antibodies against carbohydrates can be used effectively in methods of treating both lentiviral infections and malignant tumors.

We have also discovered methods of making antibodies by using one or more carbohydrate or glycan chains as epitopes.

Accordingly, in one embodiment, the present invention provides methods of treating lentivirus infections, such as primate lentivirus infections, e.g., HIV-1, by using antibodies directed against glycans present on the lentivirus surface glycoprotein. Preferably one uses monoclonal antibodies, more preferably humanized antibodies. Still more preferably, one uses a Fab or single chain antibody.

We have discovered that antibodies, including monoclonal antibodies (mAbs) directed to egg carbohydrates of the helminth Schistosoma mansoni can neutralize lentiviral, such as primate lentiviral, e.g., HIV-1 infection mediated by primary, non-TCLA isolates, in vitro.

In one embodiment, the invention provides a method of treating malignant tumors using antibodies against carbohydrates, alone or in combination with other antigens, such as known tumor associated antigens, such as PSA, CEA, and the like.

We have discovered that a variety of tumor cells, including but not limited to CMS5, a methylcholanthrene-induced fibrosarcoma of BALB/c origin, MCA38, a murine colon carcinoma, Lewis lung carcinoma, and B16 melanoma cell lines, and CT-26 express Lewis X-containing glycans. We found that the increase in Gr1+/CD11b+ cells in tumor-bearing mice is associated with a decrease in the protective Th1 responses and an increase in the deleterious Th2 responses. Targeting the Lewis X-containing glycan an affect the downregulation of Th1 responses and induction of Th2 responses. Thus, the antibodies against these glycants can be used to treat cancers, such as solid tumors or leukemias, or lymphomas. The findings show that targeting Lewis X-containing glycans, for example using antibodies, can prevent the decreased Th1 response and thus provide a novel way to treat or provide supportive treatment of malignancies.

The lentiviruses that can be targeted using the glycan or carbohydrate targeting antibodies include lentiviruses such as bovine lentiviruses (e.g., Bovine immunodeficiency virus, Jembrana disease virus), equine lentiviruses (e.g. Equine infectious anemia virus), feline lentiviruses (e.g. Feline immunodeficiency virus), ovine/caprine lentivirus (e.g. Caprine arthritis-encephalitis virus, Ovine lentivirus, Visna virus) and primate lentivirus group. Preferably the lentiviruses are primate lentiviruses. The primate lentivirus group includes Human immunodeficiency virus (HIV) including Human immunodeficiency virus type 1 (HIV-1), Human immunodeficiency virus type 2 (HIV-2), and Human immunodeficiency virus type 3 (HIV-3), as well as Simian AIDS retrovirus SRV-1, including Human T-cell lymphotropic virus type 4 (HIV-4) and Simian immunodeficiency virus (SIV). Targeting HIV is preferred. Still more preferably one targets HIV-1.

The carbohydrates of lentivirus such as HIV-1 gp120/41 and those of S. mansoni egg antigen share a common presence of the tetrasaccharide LewisY [1].

In one embodiment, the invention provides a method of treating lentivirus, preferably primate lentivirus, such as HIV-1 infection comprising the steps of administering to an individual infected with the lentivirus, one or more antibodies, preferably monoclonal antibodies that recognizes a carbohydrate antigen and a pharmaceutically acceptable carrier. In one embodiment, a cocktail of monoclonal antibodies recognizing a different carbohydrate or glycan antigens is used.

In one preferred embodiment, the antibody is generated against carbohydrates present on the surface of S. mansoni, such as Lewis antigens. For example, an antibody such as E.5 is used.

In one preferred embodiment, the antibody is selected from the group consisting of E.5, E.1 and E.3 or any combination thereof.

In one embodiment, the structure recognized by neutralizing monoclonal antibody E.5 is Lewis X trisaccharide, such as LNFPIII.

Because each of these monoclonal antibodies independently neutralizes HIV-1, they can all be used singly, or in combination as therapeutic products to eliminated virus and virally infected cells in vivo.

In one embodiment, the invention provides a method of inhibiting a lentivirus, preferably a primate lentivirus such as HIV-1 comprising the steps of administering to an individual affected with HIV-1, a fusion molecule comprising an antibody, preferably a monoclonal antibody that is fused to a drug, toxin or radionuclide, which can kill the infected cells that the monoclonal antibodies bind to.

In one preferred embodiment, the individual infected with the primate lentivirus, such as HIV-1 include individuals known to have acute infection, individuals refractory to drug treatment regimens, and infected pregnant women prior to delivery to prevent mother to child transmission, infants born to HIV-1 infected mothers to neutralize any transmitted virus and thereby reduce chance of infection.

In one preferred embodiment, the neutralizing antibodies, preferably monoclonal antibodies are administered to HIV-1 positive mothers who are breast feeding their children to reduce risk of mother to child transmission via breast milk.

We have also discovered that in addition to HIV-1, E.5 binds to Lewis X, which is also found on several different tumor cells and cancers. Therefore, in one embodiment, E.5 monoclonal antibody is be used as a therapeutic agent to target cancer cells, again, using either drug, toxin or radionuclide conjugated E.5 to target and kill tumor/cancer cells.

The structure of glycans recognized by E.1 and E.3 monoclonal antibodies remain unidentified and therefore, they represent probes to determine additional glycan antigen targets to be used for construction of a glycan antigen based vaccine for HIV.-1.

Accordingly, in one embodiment, the invention provides a method of preventing HIV-1 infection by vaccinating an individual using glycan antigens.

In yet another embodiment, the invention provides methods for developing antibodies that target HIV-1 by using carbohydrates as antigens.

Accordingly, the invention provides a method for treating HIV-1 infection in a mammal, the method comprising administering to a mammal infected with HIV-1 an effective amount of at least one antibody that recognizes at least one carbohydrates and a pharmaceutically acceptable carrier.

Our data also show that E.1 and E.3 bind to certain tumors/cancers and therefore these antibodies can also be used as anti-tumor/cancer/malignant cell therapeutic reagents.

Accordingly, in one embodiment, the invention provides method of treating malignant tumors by administering to an individual in need thereof, one or more antibodies agains carbohydrates.

In one embodiment, the method is used as a combination therapy where an individual also receives other tumor treatments, such as vaccination with a tumor specific antigen.

In one embodiment, the antibody is selected from the group consisting of E.5, E.1, and E.3 or any combination thereof.

In one embodiment, the carbohydrate comprises Lewis X trisaccharide.

In one preferred embodiment, the antibody is a monoclonal antibody.

In one embodiment, the antibody is a single chain antibody.

In one embodiment, the antibody comprises a constant region of human origin.

In one preferred embodiment, the antibody is a humanized antibody, a humanized chimeric antibody or the antibody comprises human variable regions.

In one embodiment, the antibody is an immunologically active antibody fragment. Preferably, the fragment is selected from the group consisting of Fab, F(v), Fab′ and F(ab)₂ fragment.

The invention further provides a method for preventing or inhibiting HIV-1 infection in a mammal, comprising the steps of administering to an individual at least one antigen comprising a carbohydrate in a pharmaceutically acceptable carrier.

In one preferred embodiment, the antigen comprises Lewis X trisaccharide.

### DETAILED DESCRIPTION OF THE INVENTION

We have discovered that targeting carbohydrates on cell or viral particle surfaces by using antibodies against such carbohydrates can be used in methods of treating lentiviral infections and malignant tumors.

One can “target” a carbohydrate by known means such as using a compound that binds to the carbohydrate. This can include antibodies, small molecules, carbohydrate-binding peptides, such as lectins, and the like.

The term “antibody” as used herein and throughout the specification is meant to refer to an immunoglobulin protein that is capable of binding an antigen. Antibody as used herein is meant to include antibody fragments, e.g. F(ab′)2, Fab′, Fab, single chain antibodies, dAbs (heavy chain portions) capable of binding the antigen or antigenic carbohydrate fragment of interest. Preferably, the binding of the antibody to the carbohydrate antigen, such as Lewis X trisaccharide.

The term “humanized antibody” is used herein to describe complete antibody molecules, i.e. composed of two complete light chains and two complete heavy chains, as well as antibodies consisting only of antibody fragments, e.g. Fab, Fab′, F (ab′)2, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human patient.

The term “chimeric antibody” is used herein to describe an antibody molecule as well as antibody fragments, as described above in the definition of the term “humanized antibody.” The term “chimeric antibody” encompasses humanized antibodies. Chimeric antibodies have at least one portion of a heavy or light chain amino acid sequence derived from a first mammalian species and another portion of the heavy or light chain amino acid sequence derived from a second, different mammalian species.

One can use, for example, phage libraries comprised of human variable chains to screen for appropriate antibodies.

Antibodies (monoclonal or polyclonal) are commercially available and may also be prepared by methods known those of skill in the art, for example, in Current Protocols in Immunology, John Wiley & Sons, Edited by: John E. Coligan, Ada M. Kruisbeek, David H. Margulies, Ethan M. Shevach, Warren Strober, 2001.

Neutralizing antibodies are readily raised in animals such as rabbits or mice by immunization with one or more carbohydrate. Immunized mice are particularly useful for providing sources of B cells for the manufacture of hybridomas, which in turn are cultured to produce large quantities of anti-HIV-1 monoclonal antibodies that recognize carbohydrates present on the surface of the HIV-1 virus envelope. Preferably, both regions and the combination have low immunogenicity as routinely determined. Humanized antibodies are immunoglobin molecules created by genetic engineering techniques in which the murine constant regions are replaced with human counterparts while retaining the murine antigen binding regions. The resulting mouse-human chimeric antibody should have reduced immunogenicity and improved pharmacokinetics in humans. Preferred examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

In one preferred embodiment, the antibodies are selected from the group consisting of E.5, E.1 and E.3. The preparation of these antibodies has been described in detail in a publication “Schistosoma mansoni: anti-egg monoclonal antibody protect s against cercarial challenge in vitro by Ham et al. J. Exp. Med. 159:1371-1387, 1984, the content of which is herein incorporated by reference in its entirety.

The identity of the glycan structures bound by mAbs E.1 and E.3 can be determined by a skilled artisan by testing for binding of each of these mAbs to glycan structures plated on glycan arrays, including a gp120 array.

One can readily screen for other antibodies, and carbohydrate-binding molecules against the carbohydrate or glycan antigens, such as Lewis X trisaccharide. One can then screen for the desired effect in carbohydrate binding of these carbohydrate-binding molecules using methods such as those described in Examples 1-3, and incorporated herein by reference.

Antibodies of the invention are preferably substantially pure when used in the disclosed methods and assays. References to an antibody being “substantially pure” mean an antibody or protein which has been separated from components which naturally accompany it.

In one embodiment, the invention provides co-administration of co-stimulatory molecules with one or more of the glycan antigens, such as LFNPIII. Co-stimulatory molecules are known in the art and include B7 and other CD4+ and CD8+ activators. Preferably, one uses co-administration of immune modulating molecules, such as LFA-3, ICAM-1, and B7. In one preferred embodiment, one uses a combination of the co-stimulatory molecules. The invention also provides co-administration of granulocyte macrophage-stimulating factor (GM-CSF) with one or more of the glycan antigens, such as LNFPIII. In one embodiment, cytokines, such as IL-2, IFN-gamma, IFN-alpha, or IFN-beta are used a co-stimulatory molecules.

In one preferred embodiment, Hsp70 is used as a co-stimulatory molecule to enhance the immune response to glycan antigens. For example, Hsp70 can be used incombination with LNFPIII, E.1 and E.3.

In one embodiment, the method comprises administering to an individual an initial “prime” with a composition containing one or more glycan antigen, followed by one or preferably multiple “boosts” with a composition containing one or more of the glycan antigens. One can also use one or more of the above-listed co-stimulatory molecules in the prime and boost injections.

Methods of Treating Lentiviral Infections Using Antibodies Against Carbohydrates

The tested anti-S. mansoni mAbs react with fucose-containing epitopes [17, 29, of Example 1], and 90% of the gp120/41 glycosylation sites are fucosylated [19, of Example 1].

We have found that antibodies against the carbohydrates present on the surface of S. mansoni can also vigorously inhibit isolates of HIV-1 characteristically resistant to HIV-1 antibody-positive human sera, regardless of the strain's preferred coreceptor usage.

We have discovered that antibodies against glycan or oligosaccharide targets or epitopes are acpable of efficiently neutralizing lentiviruses, preferably primate lentiviruses. For example, we can neutralize primary isolates of HIV-1, including primary HIV-1 isolates and laboratory HIV-1 strains. The targets also include both R5-tropic and X4-tropic as well as R5X4 duotropic primary isolates of HIV-1.

Preferably, the antibodies are anti-glycan monoclonal antibodies that broadly neutralize lentiviruses, such as HIV-1, although polyclonal antibodies can also be used.

Preferably, the antibody is generated against glycan epitopes present on cellular receptors used by lentiviruses to bind to the cells. In one preferred embodiment, the epitope is LNFPIII, which is a glycan that binds to a cellular receptor DC-SIGN, which is used, e.g., by HIV.

In one preferred embodiment, one includes heat shock protein-70 (Hsp70) with the S. mansoni vaccine.

For example, lentiviruses are a family of retroviruses that includes bovine lentiviruses (e.g., bovine immunodeficiency virus, Jembrana disease virus), equine lentiviruses (e.g. equine infectious anemia virus), feline lentiviruses (e.g. feline immunodeficiency virus), ovine/caprine lentivirus (e.g. caprine arthritis-encephalitis virus, ovine lentivirus, visna virus) and primate lentivirus group. The primate lentivirus group includes human immunodeficiency virus (HIV) including human immunodeficiency virus type 1 (HIV-1), human immunodeficiency virus type 2 (HIV-2), and human immunodeficiency virus type 3 (HIV-3), as well as simian AIDS retrovirus SRV-1, including human T-cell lymphotropic virus type 4 (HIV-4) and simian immunodeficiency virus (SIV).

The human immunodeficiency virus (HIV-1, also referred to as HTLV-III, LAV or HTLV-III/LAV) is the etiological agent of the acquired immune deficiency syndrome (AIDS) and related disorders. (Barre-Sinoussi, et al., Science, 220:868-871 (1983); Gallo, et al., Science, 224:500-503 (1984); Levy, et al., Science, 225:840-842 (1984); Popovic, et al., Science, 224:497-500 (1984); Sarngadharan, et al., Science, 224:506-508 (1984); Siegal, et al., New England Journal of Medicine, 305:1439-1444 (1981)). This disease is characterized by a long asymptomatic period followed by the progressive degeneration of the immune system and the central nervous system. Studies of the virus indicate that replication is highly regulated, and both latent and lytic infection of the CD4 positive helper subset of T-lymphocytes occur in tissue culture (Zagury, et al., Science, 231:850-853 (1986)). The expression of the virus in infected patients also appears to be regulated as the titer of infectious virus remains low throughout the course of the disease. Molecular studies of the replication and genomic organization of HIV-1 show that it encodes a number of genes (Ratner, et al., Nature, 313:277-284 (1985); Sanchez-Pescador, et al., Science, 227:484-492 (1985); Muesing, et al., Nature, 313:450-457 (1985); Wain-Hobson, et al., Cell, 40:9-17 (1985)). Three of the genes, the gag, pol and env genes are common to all retroviruses. The genome also encodes additional genes that are not common to most retroviruses, the tat, rev (formerly referred to as art), nef, vif, vpr and vpu genes (Sodroski, et al., Science, 231:1549-1553 (1986); Arya, et al., Science, 229:69-73 (1985); Sodroski, et al., Nature, 321:412-417 (1986); Feinberg, et al., Cell, 46:807-817 (1986); Haseltine, Journal of Acquired Immune Deficiency Syndrome, 1:217-240 (1988); Cohen, et al., Nature, 334:532-534 (1988); Wong-Staal, et al., AIDS Res. and Human Retro Viruses, 3:33-39 (1987)).

Nucleotide sequences from viral genomes of other retroviruses, particularly HIV-2 and simian immunodeficiency viruses, SIV (previously referred to as STLV-III), also contain the structural genes including env as well as regulatory sequences such as tat, rev and nef (Guyader, et al., Nature, 326:662-669 (1987); Chakrabarti, et al., Nature, 328:543-547 (1987)). These three HIV viruses share a similar genetic organization, even though there can be sequence variations.

The env genes of HIV-1, HIV-2 and SIV all produce an envelope glycoprotein, which is cleaved, with one portion being an exterior viral envelope protein subunit referred to as gp120. The binding and fusion of HIV-1, HIV-2 and SIV viruses with cells is mediated by specific interaction between the external subunit of this gp120 viral envelope protein and the CD4 receptor on the target cell surface (Dalgleish, et al., Nature, 312:763-767 (1984); Klatzmann, et al., Nature, 312:767-768 (1984); Berger, et al., PNAS, 85:2357-2361 (1988)).

Serum antibodies reacting with the HIV-1 gp120 can neutralize viral infection by binding to several sites on the molecule (Haigwood, et al., Vaccines, 90:313-320 (1990); Steimer, et al., Science, 254:105-108 (1991)). The earliest neutralizing human antibody response is directed to epitopes in the third hypervariable region of gp120, the principle neutralizing domain, which is contained within a loop formed by disulfide bonding (Rusche, et al., PNAS, 85:3198-3202 (1988); Goudsmit, et al., PNAS 85:4478-4482 (1988); Palker, et al., PNAS, 85:1932-1936 (1988)). These antibodies are frequently strain-specific (Kang, et al., PNAS, 88:6171-6175 (1991)). Envelope glycoprotein variation both within the linear epitope and outside the epitope can allow escape of viruses from neutralization by these antibodies (McKeating, et al., AIDS, 3:777-783 (1989); Looney, et al., Science, 241:357-360 (1988)). Later in the course of HIV infection, more broadly neutralizing antibodies appear (Ho, et al., J. Viro., 61:2024-2028 (1987)). A large fraction of these broadly neutralizing antibodies, which are present in low concentrations in patients' sera, are directed to conformationally sensitive epitopes on gp120 (Nara, et al., J. Viral., 61:3173-3180 (1987); Goudsmit, et al., Vaccine, 6:229-238 (1988)). A subset of the broadly reactive antibodies, found in the serum of patients, interferes with the binding of gp120 and CD4 (Steimer, et al., supra; Rusche, et al., supra; McDougal, et al., J. Immunol., 137:2937-2944 (1986); Ardman, et al., J. AIDS, 3:206-214 (1990); Sclmittman, et al., J. Immunol., 141:4181-4186 (1988)). These antibodies appear to be reactive with a discontinuous epitope on gp120 which encompasses the CD4 binding region (Olshevsky, et al., J. Virol., 64:5701-5707 (1990); Thali, et al., J. Virol., 65:6188-6193 (1991)). This region of gp120 is well-conserved, although not invariant.

According to an estimate of the Joint United Nations Program on HIV/AIDS (UNAIDS), there are approximately 40 million people living with HIV-1/AIDS globally. The worldwide AIDS epidemic is most devastating in developing countries (Esparza J, Bhamarapravati N. Accelerating the development and future availability of HIV-1 vaccines: why, when, where, and how? Lancet 2000; 355: 2061-2066; Essex M. State of the HIV pandemic. J Hum Virol 1998; 1: 427-429; UNAIDS. Posting Update. AIDS epidemic update. December. 2001). HIV-1 infection is epidemic in India, China, South America, the horn of Africa, and sub-Saharan Africa (Esparza J, Bhamliarapravati N. Accelerating the development and future availability of HIV-1 vaccines: why, when, where, and how? Lancet 2000; 355: 2061-2066; Essex M. State of the HIV pandemic. J Hum Virol 1998; 1: 427-429; Janssens W, et al., Aids 1997; 11: 705-712; Shafer R W, et al., J Virol 1997; 71: 5441-5448; Staneki K A and Way PO. 1996. Aids in the World II, Vol. 16. Oxford Express. New York; Weniger B G, et al., Aids 1994; 8 Suppl 2: S13-28). However, the majority of countries with the highest HIV-1 prevalence are in southern Africa (UNAIDS. Posting Update. AIDS epidemic update. December. 2001; UNAIDS. Programme Coordinating Board. Ninth Meeting. Report of the Executive Director. 2000). AIDS continues to be a devastating source of morbidity and mortality throughout Africa and other developing countries. Although a number of methods have been developed for treating people infected with HIV, such as “cocktail” treatments, these methods have not proved entirely successful and the method of treatment is complicated. The rapid expansion of the AIDS epidemic has had and will continue to have a significant impact on the future development of numerous countries in southern Africa, India, and China. Without a means of slowing the spread of HIV-1 in these regions the impact will only worsen. Therefore developing means for treating individuals with HIV that are simple and inexpensive are desired. One method is to enhance an immune response such as with a vaccine.

While stimulating immune responses such as with an AIDS vaccine is desired, no clear consensus opinions of investigators as to the type of immune response considered most important in eliciting a protective AIDS vaccine has been reached (Cohen J. Science 2000; 287: 1567; Lee T N. Acquired immunodeficiency Disease vaccines: design and development, p 605-616. In V. T. J. DeVita, and e. al. (eds), AIDS: Etiology, Diagnosis, Treatment and prevention, Fourth edition ed. Lippincott-Ravin, Philadelphia-New York. 1997; NIAID. HIV vaccine development status report May 2000, The NIAID Division of AIDS. 2000; Berman P W. AIDS Res Hum Retroviruses 1998; 14 Suppl 3: S277-289; Berman P W, et al., Virology 1999; 265: 1-9; Francis D P, et al. AIDS Res Hum Retroviruses 1998; 14 Suppl 3: S325-331; Migasena S, et al. AIDS Res Hum Retroviruses 2000; 16: 655-663; Betts M R, et al., AIDS Res Hum Retroviruses 1999; 15: 1219-1228; Borrow P, et al., J Virol 1994; 68: 6103-6110; Greenough T C, et al., J Infect Dis 1997; 176: 118-125; Doling A M, et al., Infect Immun 1999; 67: 3290-3296; Jin X, et al., J Exp Med 1999; 189: 991-998; Ogg G S, et al., Aids 1998; 12: 1561-1563; Ogg G S, et al., J Virol 1999; 73: 9153-9160; Pontesilli O, et al., Immunol Lett 1997; 57: 125-130; Pontesilli O, et al., J Infect Dis 1998; 178: 1008-1018; Schmitz J E, et al., Science 1999; 283: 857-860). Initially, vaccines were designed to elicit neutralizing antibodies and the Vaxgen gp120 based vaccine came out of such studies (Cohen J. Science 2000; 287: 1567; Lee T N. Acquired immunodeficiency Disease vaccines: design and development, p 605-616. In V. T. J. DeVita, and e. al. (eds), AIDS: Etiology, Diagnosis, Treatment and prevention, Fourth edition ed. Lippincott-Ravin, Philadelphia-New York. 1997; NIAID. HIV vaccine development status report May 2000. The NIAID Division of AIDS. 2000; Berman P W. AIDS Res Hum Retroviruses 1998; 14 Suppl 3: S277-289; Berman P W, et al., Virology 1999; 265: 1-9; Francis D P, et al. AIDS Res Hum Retroviruses 1998; 14 Suppl 3: S325-331; Migasena S, et al. AIDS Res Hum Retroviruses 2000; 16: 655-663).

Attempts to produce vaccines that elicited both humoral and cellular immune responses have been made and currently there are several candidate vaccines either in testing, or about to enter testing. These vaccines are based on inducing neutralizing antibodies or eliciting CTL responses (Migasena S, et al., AIDS Res Hum Retroviruses 2000; 16: 655-663; Ferrantelli F and Ruprecht R M, Curr Opin Immunol 2002; 14: 495-502; Xu W, Hofmann-Lehmann R, et al., Vaccine 2002; 20: 1956-1960; Hofmann-Lehmann R, et al. J Med Primatol 2001; 30: 190-196; Moulard M, et al., Proc Natl Acad Sci USA 2002; 99: 6913-6918; Kwong P D, et al., Nature 2002; 420: 678-682; Cox W I, et al., Virology 1993; 195: 845-850; Turner J L, et al., HIV Med 2001; 2: 68-77; Mwau M, McMichael A J., J Gene Med 2003; 5: 3-10).

However, due to the difficulty in generating broadly neutralizing antibody responses, much of new research is placed on discovery of CD8+ T cell epitopes and production of single or multi-CTL epitope vaccines. This switch in emphasis is readily seen by examining candidate vaccines that are in or will enter Phase I clinical trials. These vaccines are primarily focused on eliciting CTL responses (Betts M R, et al., AIDS Res Hum Retroviruses 1999; 15: 1219-1228; Borrow P, et al., J Virol 1994; 68: 6103-6110; Greenough T C, et al., J Infect Dis 1997; 176: 118-125; Doling A M, et al., Infect Immun 1999; 67: 3290-3296; Jin X, et al., J Exp Med 1999; 189: 991-998; Ogg G S, et al., Aids 1998; 12: 1561-1563; Ogg G S, et al., J Virol 1999; 73: 9153-9160; Pontesilli O, et al., Immunol Lett 1997; 57: 125-130; Pontesilli O, et al., J Infect Dis 1998; 178: 1008-1018; Schmitz J E, et al., Science 1999; 283: 857-860).

Infection with HIV leads, in most cases, to a progressive decline in the number and functions of CD4+ T cells with the eventual appearance of clinical manifestations of cellular immunodeficiency, such as opportunistic infections and malignancies, i.e., AIDS (Fauci, et al., Ann. Int. Med., 100:92-99 (1984)). The entry of HIV-1 into the target cells requires, in association with the CD4 molecule, the simultaneous virus binding to chemokine receptors. Some viruses interact with the chemokine receptor CCR5 and are termed R5-tropic isolates, some bind to the chemokine receptor CXCR4 and are termed X4-tropic, and others are able to use either one, and are then classified as R5X4 dual-tropic viruses [2, of Example 1]. R5 viruses can be isolated from patients during the whole course of the infection, are usually associated with the asymptomatic clinical status of the HIV-1-infected patients, and are the phenotypes preferentially transmitted in vivo [33 of Example 1]. X4 and R5X4 isolates are more frequently found in patients progressing from the asymptomatic clinical status to AIDS [10 of Example 1].

An immune response against HIV-1 can be detected a few weeks after the primary infection [6 of Example 1]. Following the virus seeding in the lymphoid tissues, viral replication is controlled mainly by cytotoxic CD8+ T cells [3, 20 of Example 1]. Most patients infected by HIV-1 also mount a strong humoral immune response against the virus [6 of Example 1], but, so far, there is no clear evidence showing that the antibodies are really effective in limiting the progression of the infection to AIDS. Several studies have shown that primary viruses are remarkably resistant to neutralization by antibodies, either from vaccine sera or from serum samples of HIV-1-infected individuals [9 of Example 1]. Likewise, it has been shown that resistance to neutralizing antibodies is independent of the virus preferential coreceptor usage [7, 18, 21, 27 of Example 1]. Experimental evidence indicates that primary isolates of HIV-1 escape the humoral immune response because immunodominant epitopes on the envelope glycoprotein (gp120/41) of these viruses are not accessible to neutralizing antibodies [4, 25 of Example 1]. In contrast, tissue culture laboratory-adapted (TCLA) isolates of HIV-1 are sensitive to neutralizing antibodies, most likely because immunodominant epitopes are more exposed to immunoglobulin binding [28 of Example 1].

The extensive glycosylation of gp120/41 may hide immunodominant peptidic epitopes, preventing the binding of neutralizing antibodies [23, 32 of Example 1]. However, these carbohydrates may also function as neutralization epitopes, since monoclonal antibodies (mAbs) against oligosaccharides inhibited HIV-1 infection in cell-free virus experiments, as well as in syncytium-inducing assays [13, 14, 24, 26 of Example 1].

Although patients infected with lentiviruses, such as HIV-1, eventually develop a potent humoral immune response against the virus, but HIV-1 primary isolates are remarkably resistant to neutralizing antibodies. Accordingly, a need to develop novel ways to attack the virus continues to exist.

The present invention provides anti-glycan antibodies, preferably anti-glycan monoclonal antibodies (mAbs) that independently neutralize primary isolates of HIV-1.

In one preferred embodiment, the invention provides a glycan antigen pentasaccharide called LNFPIII. This glycan binds to the cellular receptor DC-SIGN, which HIV uses to bind to cells and presents a potential mechanism as to how this mAb is able to neutralize virus. In one preferred embodiment, one includes of Hsp 70 with the primate lentivirus, such as HIV-1 CTL or Schistosoma mansoni vaccines which enhances the antigen-specific IgG antibody titers in the recipient subject individual.

Preferred antibodies useful in the methods of the invention are monoclonal antibodies, preferably humanized. One preferred antibody is a monoclonal antibody to LNFPIII, such as E.5, a monomeric IgM which recognizes a defined epitope called Lewis X trisaccharide. Other preferred antibodies include, but are not limited to E.1, an IgG2b class antibody, and E.3, an IgG3 class antibody. These are also antibodies directed against distinct oligosaccharide targets and have capasity to neutralize HIV-1. All of these antibodies bind to gp120.

One preferred antibody that is useful in the methods of the invention is an antibody against Lewis X antigen.

Accordingly, Lewis X is also one preferred antigen in the vaccines of the present invention. Lewis X is immunogenic and functions as an adjuvant for third party antigens as well as directly activates dendritic cells (DCs) through toll-like receptor-4 (TLR4) driving T-helper cell (Th) type-2 CD4+ T cell maturation. In one preferred embodiment, the invention provides a method of vaccinating a mammal against lentivirus infection, preferably primate lentivirus infection, more preferably against HIV-1 or HIV-2. The antigen useful according to the method is preferably a stimulatory form of a compound comprising a Lewis antigen, such as a compound comprising cross-linked (i.e., multivalent) Lewis^{Y} oligosaccharides, Lewis X oligosaccharides, Lewis^{A} oligosaccharides or derivatives thereof, for example, sulfated, sialylated or sulfo-sialylated forms of these oligosaccharides. The stimulatory compound can be, for example, a conjugate of the Lewis antigen and a carrier molecule, such as, human serum albumin or polyacrylamide. For stimulating responses by human immune cells, the agent preferably comprises a Lewis X oligosaccharide or a derivative thereof. Still more preferably, the agent comprises LNFPIII.

The vaccines or antigenic agents of the invention stimulate production by cells of at least one cytokine that regulates development of a Th1 or Th2 response. In a preferred embodiment, the antigenic agent is a stimulatory form of a compound comprising a Lewis antigen. In one preferred embodiment the antigen is LNFPIII. A “stimulatory form of a compound comprising a Lewis antigen” typically is one in which the carbohydrate structure is present in a multivalent, crosslinked form. In a preferred embodiment, the stimulatory form of a compound comprising a Lewis antigen is a conjugate of a carrier molecule and multiple carbohydrate molecules expressing a Lewis antigen. For example, carbohydrate molecules can be conjugated to a protein carrier, such as a conjugate of human serum albumin (HSA) and Lewis^{Y} oligosaccharides (referred to herein as HSA-LeY). When a sugar-carrier protein conjugate is to be administered to a subject, the carrier protein should be selected such that an immunological reaction to the carrier protein is not stimulated in the subject (e.g., a human carrier protein should be used with a human subject, etc.). Alternative to a carrier protein, multiple Lewis antigens can be conjugated to other carrier molecules, such as a solid support, such as beads (e.g., polyacrylamide, agarose, SEPHAROSE™ polystyrene and the like) or a plate. The degree of stimulatory ability of the conjugate is influenced by the density of sugars conjugated to the carrier (see, e.g., Example 4 in U.S. Pat. No. 6,540,999). Preferably, the sugar molecules comprise at least 10% of the conjugate by weight, more preferably at least 15% of the conjugate by weight, even more preferably at least 20% of the conjugate by weight and even more preferably at least 25% of the conjugate by weight. In certain embodiments, the sugar molecules comprise about 10-25% of the conjugate by weight, about 15-25% of the conjugate by weight or about 20-25% of the conjugate by weight. In a preferred embodiment, the stimulatory form of a compound comprising a Lewis antigen is a conjugate of multiple carbohydrate molecules expressing a Lewis antigen and the carrier polyacrylamide. More preferably, the polyacrylamide conjugates comprise 25 to 30 (or more) sugars/conjugate, wherein the average molecular weight of the conjugate is approximately 30 kD.

The Lewis antigens present in the conjugate can be, for example, LewisY, Lewis X, Lewis^{A} or Lewis^{B} oligosaccharides, or derivatives thereof. In one preferred embodiment, the antigen is LNFPIII. In certain embodiments for stimulation of human cells, the stimulatory agent preferably comprises LewisY oligosaccharides or derivatives thereof. Within the stimulatory agent, the Lewis antigen can be present within a larger carbohydrate structure. For example, the carbohydrate portion of the stimulatory agent can be lacto-N-fucopentaose III (LNFP-III), which has the structure: {Galβ(1-4)[Fuc(α1-3)]GlcNac(β1-3)Gal(β1-4)Glc} and comprises the Lewis X oligosaccharide, or lacto-N-difucohexose I (LND), which has the structure: {Fuc(α1-2) Gal(β1-3)[Fuc(α1-4)]GlcNac(β1-3)Gal(β1-4)Glc} and comprises the Lewis^{B} oligosaccharide. Other related carbohydrates comprising Lewis antigens that are suitable for use in a stimulatory agent of the invention will be apparent to those skilled in the art.

In addition to conjugates comprising Lewis antigen-containing sugars described above, another form of a stimulatory agent comprising a Lewis antigen is an isolated protein that naturally expresses Lewis antigens in a form suitable for stimulatory activity. One example of such a protein is schistosome egg antigen (SEA), which expresses the Lewis X oligosaccharide. Other proteins that have been reported to express Lewis antigens include tumor-associated antigens (see e.g., Pauli, B. U., et al. (1992) Trends in Glycoscience and Glycotechnology 4:405-414; Hakomori, S-I. (1989) Adv. Cancer Res. 52:257-331) and HIV gp120 (Adachi, M., et al. (1988) J. Exp. Med 167:323-331).

Stimulatory agents for use in the methods of the invention can be purchased commercially or can be purified or synthesized by standard methods. Conjugates of Lewis antigen-containing sugars and a carrier protein (e.g., HSA) are available, for example, from Accurate Chemicals, Westbury, N.Y. Conjugates of Lewis antigen-containing sugars and polyacrylamide are available from GlycoTech, Rockville, Md. Schistosome egg antigen (SEA) can be purified from Schistosoma mansoni eggs as described in Ham, D. H., et al. (1984) J. Exp. Med. 159:1371-1387. Lewis antigen-containing sugars, or derivatives thereof, can be conjugated to a carrier protein or solid support (e.g., beads or a plate) by standard methods, for example using a chemical cross-linking agent. A wide variety of bifunctional or polyfunctional cross-linking reagents, both homo- and heterofunctional, are known in the art and are commercially available (e.g., Pierce Chemical Co., Rockford, Ill.).

The ability of a stimulatory antigenic agent of the invention to stimulate production by immune cells of at least one cytokine that regulates a Th1 or Th2 response can be evaluated using an in vitro culture system such as that described in the U.S. Pat. No. 6,540,999 (e.g., peripheral blood mononuclear cells) are cultured in the presence of the stimulatory agent to be evaluated (e.g., at a concentration of 100 μM for sugar conjugates) in a medium suitable for culture of the chosen cells. After a period of time (e.g., 24-72 hours), production of a cytokine that regulates development of a Th1 or Th2 response is assessed by determining the level of the cytokine in the culture supernatant. Preferably, the cytokine assayed is IL-10. Additionally or alternatively, IL-4 and/or PGE₂ levels can be assessed. Cytokine levels in the culture supernatant can be measured by standard methods, such as by an enzyme linked immunosorbent assay (ELISA) utilizing a monoclonal antibody that specifically binds the cytokine. An ELISA for measuring IL-10 levels is described further in Kullberg, M. C., et al. (1992) J. Immunol. 148:3264-3270. An ELISA kit for measuring PGE₂ levels is commercially available from, e.g., Advanced Magnetics, Cambridge, Mass. The ability of a stimulatory agent to stimulate cytokine production is evidenced by a higher level of cytokine (e.g., IL-10) in the supernatants of cells cultured in the presence of the stimulatory agent compared to the level of cytokine in the supernatant of cells cultured on the absence of the stimulatory agent.

As used herein, the term “Lewis antigen” is intended to include carbohydrates having as a core sequence either the lacto type I structure {Gal(β1-3)GlcNac} or the lacto type II structure {Gal(β1-4)GlcNac}, substituted with one or more fucosyl residues. The Lewis antigen may comprise a single substituted core sequence or a repetitive series of substituted core sequences. Moreover, the core sequence may be present within a larger sugar. Accordingly, a Lewis antigen-containing oligosaccharide can be, for example, a trisaccharide, a tetrasaccharide, a pentasaccharide, and so on. Types of Lewis antigens include Lewis X, Lewis^{Y}, Lewis^{A} and Lewis^{B} oligosaccharides and derivatives thereof. Synthetic structural homologues of these carbohydrates that retain the immunomodulatory capacity described herein are also intended to be encompassed by the term “Lewis antigen”.

As used herein, the term “Lewis X oligosaccharide” refers to a lacto type II carbohydrate comprising the structure: {Gal(β1-4)[Fuc(α1-3)]GlcNac)}.

As used herein, the term “Lewis^{Y} oligosaccharide” refers to a lacto type II carbohydrate comprising the structure: {Fuc(α1-2)Gal(β1-4)[Fuc(α1-3)]GlcNac}.

As used herein, the term “Lewis^{A} oligosaccharide” refers to a lacto type I carbohydrate comprising the structure: {Gal(β1′-3)[Fuc(α1-4)]GlcNac}.

As used herein, the term “Lewis^{B} oligosaccharide” refers to a lacto type I carbohydrate comprising the structure: {Fuc(α1-2)Gal(β1-3)[Fuc(α1-4)]GlcNac}.

For example, the carbohydrate portion of the antigen can be lacto-N-fucopentaose III (LNFP-III), which has the structure: {Gal(β1-4)[Fuc(α1-3)]GlcNac(β1-3)Gal(, 1-4)Glc} and comprises the Lewis X oligosaccharide, or lacto-N-difucohexose I (LND), which has the structure: {Fuc(α1-2)Gal(β1-3)[Fuc((α1-4)]GlcNac(β1-3)Gal(α1-4)Glc} and comprises the LewisB oligosaccharide.

As used herein, a “derivative” of a Lewis oligosaccharide refers to a Lewis oligosaccharide having one or more additional substituent groups. Examples of derivatives include terminally sialylated forms of Lewis oligosaccharides (e.g., sialyl-Lewis X, sialyl-Lewis^{Y}, sialyl-Lewis^{A}, sialyl-Lewis^{B}), sulfated forms of Lewis oligosaccharides and sulfo-sialylated forms of Lewis oligosaccharides.

In one preferred embodiment, the invention provides a method of stimulating a lentivirus, preferably primate lentivirus, more preferably HIV-1 or HIV-2 specific immune response in a subject, preferably human, comprising: administering to the subject an agent comprising a Lewis antigen, such that a specific immune response to the lentivirus is stimulated in the subject. In various embodiments, the agent comprises a Lewis^{Y} oligosaccharide or a derivative thereof, a Lewis X oligosaccharide or a derivative thereof, a Lewis^{A} oligosaccharide or a derivative thereof, or a Lewis^{B} oligosaccharide or a derivative thereof. The agent can be administered, for example, intranasally, orally, intravenously, intramuscularly, subcutaneously or mucosally.

In one preferred embodiment, the antigen is the pentasaccharride Lacto-N-fucopentaose III (LNFPIII), which contains the Lewis X trisaccharride, a ligand for DC-SIGN (see, e.g. FIG. 10A).

In one embodiment, the invention provides a kit for vaccinating a human against a lentivirus, preferably HIV-1 or HIV-2 infection comprising Lewis X trisaccharide and pharmaceutical carrier, packaged with instructions for use of the pharmaceutical composition to vaccinate against lentiviral, preferably HIV-1 or HIV-2 infection.

Additionally, the method of inhibiting HIV-1 infection according to the present invention can be used in combination with other HIV-1 infection alleviating therapies well known to one skilled in the art.

Antibodies of the invention also can be used as a carrier for drugs, particularly pharmaceuticals targeted against HIV-1 infection, such as antisense molecules or siRNA molecules targeting HIV-1 transcripts.

In some instances, it may be desirable to modify the antibody of the present invention to impart a desirable biological, chemical or physical property thereto. More particularly, it may be useful to conjugate (i.e. covalently link) the antibody to a pharmaceutical agent.

Antibodies useful according to the methods of the invention also can be conjugated to a variety of other pharmaceutical agents in addition to those described above such as, e.g., drugs, enzymes, hormones, chelating agents capable of binding a radionuclide, as well as other proteins and polypeptides useful for diagnosis or treatment of HIV-1 infection. Heat shock proteins (Hsps) are essential for cellular processes, such as protein folding, protection of proteins from denaturation, aggregation and to facilitate protein transport through protein channels (Hartl F U, Nature 1996; 381: 571-579; Srivastava P K and Amato R J, Vaccine 2001; 19: 2590-2597; Kiang J G and Tsokos G C, Pharmacol Ther 1998; 80: 183-2010. Recently, Srivastava and colleagues demonstrated that recombinant Hsps also act as carriers of antigenic peptides derived from tumor cells and virus-infected cells (Srivastava P K, et al., Immunity 1998; 8: 657-665). This observation led to a number of studies examining Hsps in immunity (Srivastava P K and Amato R J, Vaccine 2001; Srivastava P K, et al., Immunity 1998; 8: 657-665; Binder R J, et al., J Immunol 2000; 165: 6029-6035; Binder R J, et al., J Immunol 2001; 166: 4968-4972; Wallin R P, et al., Trends Immunol 2002; 23: 130-135; Singh-Jasuja H, et al., Biol Chem 2001; 382: 629-636. The recognition and uptake of Hsp-peptide complexes was recently shown to be mediated by receptors on the surface of APCs (Binder R J, et al., J Immunol 2001; 166: 4968-4972; Singh-Jasuja H, et al., Biol Chem 2001; 382: 629-636; Binder R J, et al., Nat Immunol 2000; 1: 151-155; Basu S, et al., Immunity 2001; 14: 303-313; Delneste Y, et al., Immunity 2002; 17: 353; Asea A, et al., Nat Med 2000; 6: 435-442). Macrophages and dendritic cells stimulated with gp96, Hsp90 or Hsp70, secreted cytokines and upregulated expression of MHC and co-stimulatory molecules, regardless of the peptides associated with them (Binder R J, et al., J Immunol 2000; 165: 6029-6035; Asea A, et al., Nat Med 2000; 6: 435-442; Basu S, et al., Int Immunol 2000; 12: 1539-1546; Basu S and Srivastava P K, Cell Stress Chaperones 2000; 5: 443-451; Panjwani N N, et al., J Immunol 2002; 168: 2997-3003).

Hsp70 has been shown to directly induce the production of cytokines from monocytes and macrophages and also enhance NK cell proliferation and cytotoxicity whereas Hsc70 does not (Asea A, et al., Nat Med 2000; 6: 435-442; Todryk S M, et al., Immunology 2000; 99: 334-33; Fernandez N C et al., Nat Med 1999; 5: 405-411; Multhoff G, et al., J Immunol 1997; 158: 4341-4350). Human recombinant Hsp70 binds immature DCs and induces their maturation (Wang Y, et al., J Immunol 2002; 169: 2422-2429; Kuppner M C, et al., Eur J Immunol 2001; 31: 1602-1609). Similar to human, murine Hsp70 and another stress protein, gp96, induce DC maturation coincident with downregulation of the Hsp96 receptor (CD91) on mature DCs (Binder R J, et al., Nat Immunol 2000; 1: 151-155; Basu S, et al., Int Immunol 2000; 12: 1539-1546; Singh-Jasuja H, et al., Eur J Immunol 2000; 30: 2211-2215; Singh-Jasuja H, et al., Cell Stress Chaperones 2000; 5: 462-470). A recent report demonstrated that the C-terminal, peptide-binding portion of Mycobacterium tuberculosis Hsp70 stimulated Th1-polarizing cytokines, C-C chemokines, and had an adjuvant function (Wang Y, et al., J Immunol 2002; 169: 2422-2429). This C-terminal portion of Hsp70 stimulated human monocytes to produce IL-12, TNF-α, NO, and enhanced the production of IL-12 and RANTES in macaques and induced higher serum IgG2a and IgG3 Abs in mice (Wang Y, et al., J Immunol 2002; 169: 2422-2429). Further, Lehner et al. (Eur J Immunol 2000; 30: 2245-2256) demonstrate that Hsp70 linked to SIV peptides dramatically enhances γδ T cell responses and they state that linking Hsp70 to viral antigens represents a novel strategy to enhance viral-specific T cell responses. Taken together, these studies show that inducible Hsp70 activates immature DCs as well as macrophages in a receptor mediated fashion leading to enhanced Type 1 T cell responses.

Accordingly, the present invention also provides methods to use a conjugate vaccine that includes a dendritic cell targeting domain, such as Heat Shock Protein 70 in eliciting immune response to lentiviruses, such as HIV-1. In one embodiment, the immunogenicity of envelope glycans is enhanced by producing vaccines comprised of LNFPIII, E.1 and E.3 glycans conjugated to tetanus toxin T (TT) cell epitope carrier protein. TT as a carrier has a great advantage in that almost all individuals have been vaccinated with it and maintain strong, long-term memory T cell responses. For example, the invention provides an Hsp70 containing constructs for an HIV-1 CTL vaccine and in addition, a Schistosoma mansoni plasmid DNA vaccine. In both of these specific cases, we have demonstrated that both T cell and IgG antibody responses are dramatically enhanced when Hsp70 coding regions are added. Accordingly, in one preferred embodiment, the conjugates are LNFPIII-TT or LNFPIII-TT-Hsp70.

The invention also provides a kit comprising the glygan or carbohydrate antigen or combination of such antigens in a pharmaceutically acceptable carrier, and an instruction manual directing one to use said antigen(s) to immunize against a primate lentivirus, such as HIV-1 infection in one or more dosages. The antigens may also be provided in dry form and separate containers with pharmaceutically acceptable carriers can be added to the kit. The kit optionally comprises injection needles in sterile packages.

The invention further provides a kit comprising glycan-targeting antibodies, such as E.5, as described above, and an instruction manual directing to use such antibodies to treat primate lentivirus, such as HIV-1 infection. Such kit can also comprise sterile diluents and instructions how to administer the antibodies. In one embodiment, the kit also comprises materials to administer the antibody, for example using intravenous injection in a pharmaceutically acceptable carrier.

Methods of Treating Malignancies Using Antibodies Against Carbohydrates

The invention further provides methods of using antibodies against carbohydrates to treat malignancies.

We discovered that by blocking carbohydrates on the surface of malignant tumor cells one can prevent downregulation of the Th1 response to malignancies and not induce Th2 responses. This extends the effective time of Th1 cell response. We have shown that malignant cells such as CMS5, B16, MCA38, Lewing Lung cells, and express Lewis X or Lewis X-like carbohydrates and that such molecules affect Th1 and Th2 responses. We have discovered that one can target such molecules by using a molecule that binds to the carbohydrate. These include antibodies, small molecules, and other carbohydrate binding proteins, such as lectins.

The terms “malignancy”, “tumor” and “cancer” are used interchangeably throughout the specification. The terms refer to any tumor, including but not limited to malignancies of brains, eye, mouth, throut, lip, breast, liver, pancreas, lungs, stomack, colon, bone, and blood. In one embodiment, the tumor is a solid tumor. In one embodiment, the solid tumor is colon cancer, melanoma or lung cancer, for example, Lewis lung carcinoma. We discovered that the carbohydrate-binding antibodies bind to a variety of different cells from different tumors. Accordingly, we propose that expression of carbohydrates is at least common, if not universal, among tumors.

The term “mammal” as used herein, refers to any mammal. In one embodiment, the mammal is human. In one embodiment, the mammal is a primate. In one embodiment the mammal is murine, such a mouse or a rat.

In one preferred embodiment, the antibodies of the invention are used in connection with, either before, together with or after administration of tumor targeting vaccines. Useful tumor vaccines, that can be combined with the antibody therapy of the present invention are well known to one skilled in the art. Examples of such tumor vaccines have been described, for example, in U.S. Pat. Nos. 5,698,530; 6,165,460; and 6,319,496.

The antibodies of the invention can also be combined with methods, wherein in addition to a tumor associated antigen, the individual affected by the tumor is also administered a co-stimulatory molecule (see, e.g., U.S. Pat. No. 6,893,869).

Our understanding of the development of immune mechanisms effective at destroying tumor cells or malignancies has deepened during the past several years, providing a framework within which to study the failure of tumor rejection. Reports that tumor immunity that develops initially is lost before the tumor is destroyed, suggested to us that mechanisms exist to suppress tumor immunity even after it develops.

Previously, we reported that intradermal injection of CMS5 cells, a weakly immunogenic fibrosarcoma, induced an early anti-tumor response in parental tumor-bearing mice that waned with time (Gansbacher, B., K. Zier, B. Daniels, K. Cronin, R. Bannerji, and E. Gilboa. 1990. Interleukin 2 gene transfer into tumor cells abrogates tumorigenicity and induces protective immunity. J. Exp. Med. 172:1217-1224). Cytotoxic T lymphocytes (CTL) generated from spleen cells of mice injected 9-10 days earlier with tumor cells lysed CMS5 cells in an antigenically specific fashion. However, the tumors continued to increase in size and by day 28 cytotoxicity was no longer detectable. In addition, the mice had very low levels of the TCR^{˜} chain and p56lck, proteins involved in signal transduction, and lost the ability they had initially to generate anti-viral CTL, a Th1-dependent response (Young, M. R. I., et al., 1996, J. Immunol. 156:1916-1922; Bronte, V., et al., 1998, J. Immunol 161:5313-5320). Mice that lost anti-tumor immunity exhibited massive splenomegaly due to the infiltration of Gr1+/CD11b+myeloid cells (Tanaka, H., et al., 2002, J. Immunother. 25: 207-217). Several groups have described tumor and virus models in which Gr1+/CD11b+ cells suppressed T cell activation and/or function via mechanisms that involved reactive oxygen species, nitric oxide, and arginase (Salvadori, S., et al., 2000, J. Immunol 164:2214-2220; Young, M. R. I., et al., 1996. Suppression J. Immunol. 156:1916-1922; Bronte, V., M. et al., 1998, J. Immunol 161:5313-5320; Gabrilovich, D. I., et al., J. Immunol. 166:5398-5406; Mazzoni, A., et al., 2002, J. Immunol. 168:689-695; Rodriguez, P. C., et al., 2004, Cancer Res. 64:5839-5849). Following tumor resection, the level of Gr1+/CD11b+ cells returned to normal and, consistent with a role for these cells in suppressing immunity, anti-tumor immunity returned by 24-48 hours (Tanaka, H., et al., 2002, J. Immunother. 25: 207-217).

In schistosome-infected mice strong Th1-dependent responses develop initially, but decrease with time and are replaced by dominant Th2 responses (18). Recently, we reported that the inoculation of a conjugate of dextran or human serum albumin (HSA) and glycans containing the Lewis X trisaccharide, found on schistosome eggs, induced Gr1+/CD11b+ cells and acted as a Th2 adjuvant (Atochina, O., et al., 2001, J. Immunol. 167:4293-4302; Terrazas, L. I., 2001, J. Immunol. 167:5294-5303; Okano, M., et al., 2001, J. Immunol. 167:442-450). These results are consistent with those from several groups showing that molecules expressed by helminthes play an important role in regulating immunity to the worms. Of particular interest, given our results, was the report by McKee and Pearce that IL-10-secreting CD4+/CD25+Treg cells in schistosome-infected mice inhibited IL-12, suppressing Th1 responses and enhancing Th2 responses (McKee, A. S., and Pearce, E. J. 2004, J. Immunol. 173:1224-31).

Here we showed that malignant cells, for example, CMS5 cells, share with schistosome eggs at least some of the glycans containing the Lewis X-trisaccharide. We also showed that, as the tumor progresses, Th1 responses decrease and Th2 responses increase. Moreover, depletion of CD4+/CD25+ cells in naïve mice, before the injection of tumor cells, prevents tumor growth. Based upon our findings, and without wishing to be bound by a theory, we conclude that Lewis X-containing glycans on CMS5 cells induce Gr1+/CD11b+ cells, leading to the development of deleterious Th2 responses and promoting the loss of the protective Th1 responses, thereby favoring survival of the tumor. Accordingly, we propose a new method of treatment of tumors, by administering to an individual an antibody against the carbohydrates. Without wishing to be bound by a theory, we conclude that such antibodies block the function of the carbohydrates and prolong the host Th1 responses agains the tumor associated antigens.

Pharmaceutical Compositions

As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, transdermal or oral administration. In a preferred embodiment, the composition is formulated such that it is suitable for intravenous administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the modulators can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the agent may be coated in a material to protect it from the action of enzymes, acids and other natural conditions which may inactivate the agent. For example, the agent can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

For therapeutic applications, antibodies, fragments thereof or carbohydrate containing antigens of the invention may be suitably administered to a subject such as a mammal, particularly a human, alone or as part of a pharmaceutical composition, comprising the antibody, fragment thereof or antigen together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well know in the art of pharmacy. See, for example, Remington: The Science and Practice of Pharmacy, 20^{th} edition, by Alfonso R. Gennaro.

Such preparative methods include the step of bringing into association with the molecule, such as antibody or antigen, to be administered ingredients such as the carrier which constitutes one or more accessory ingredients. In general, the compositions comprising antibodies or antigens useful according to the methods of the invention are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Application of the subject therapeutics often will be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way. Systemic administration of a nucleic acid using lipofection, liposomes with tissue targeting (e.g. antibody) may also be employed.

It will be appreciated that actual preferred amounts of a given at least one antibody or a fragment thereof or antigen useful in the methods of the invention will vary to the particular antibody or mixture of antibodies or antigens being utilized, the particular compositions formulated, the mode of application, the particular site of administration, the patient's weight, general health, gender, etc., and other such factors that are recognized by those skilled in the art including the attendant physician or veterinarian. Optimal administration rates for a given protocol of administration can be readily determined by those skilled in the art using conventional dosage determination tests. In general, a suitable effective dose of one or more the above-described compounds, particularly when using the more potent antibodies, will be in the range of from 0.01 to 100 milligrams per kilogram of bodyweight of recipient per day, preferably in the range of from 0.01 to 20 milligrams per kilogram bodyweight of recipient per day, more preferably in the range of 0.05 to 4 milligrams per kilogram bodyweight of recipient per day. The desired dose is suitably administered once daily, or several sub-doses, e.g. 2 to 4 sub-doses, are administered at appropriate intervals through the day, in weekly or monthly intervals, or other appropriate schedule. Such sub-doses may be administered as unit dosage forms, e.g., containing from 0.05 to 10 milligrams of the above-described compound(s), per unit dosage, preferably from 0.2 to 2 milligrams per unit dosage.

### EXAMPLE 1

Patients infected with HIV-1 develop a potent humoral immune response against the virus, but HIV-1 primary isolates are remarkably resistant to neutralizing antibodies. Considering that the envelope glycoprotein of HIV-1 (gp120/41) is heavily glycosylated, we investigated whether anti-carbohydrate antibodies could inhibit HIV-1 infection in vitro. We studied the neutralizing activity of three monoclonal antibodies (mAbs) raised to carbohydrates of Schistosoma mansoni, against seven primary isolates of HIV-1. Assays were performed infecting peripheral blood mononuclear cells from normal donors with viral isolates previously treated with mAbs. Viral strains used were tropic for the coreceptors CCR5, CXCR4, and dual-tropic ones. We found that the anti-glycan mAbs vigorously inhibited HIV-1 infection, regardless of the preferential coreceptor usage of the isolate, in a dose-response manner. Importantly, five isolates were resistant to neutralization by two HIV-1 antibody-positive human sera endowed with potent anti-HIV-1 inhibitory activity. Our findings suggest that carbohydrates of the HIV-1 viral envelope may be a target of an effective humoral immune response elicited by vaccination.

Material and methods: Peripheral blood mononuclear cells (PBMCs) from healthy donors were obtained by density gradient centrifugation (Histopaque, Sigma Chemical Co., St. Louis, Mo.) from buffy coat preparations, and subsequently stimulated for 3 days with 2 g/ml phytohemagglutinin (PHA, Sigma) in RPMI 1640 medium (Sigma), supplemented with 10% fetal bovine serum (FBS, HyClone, Logan, Utah), HEPES, penicillin and streptomycin. For HIV-1 infection assays, PHA-stimulated PBMCs were further cultured in the same medium, containing 5 U/ml recombinant IL-2 (Sigma). The human astroglioma U87 cells stably transfected with CD4 and with CCR5 or with CXCR4 (U87-CD4+ CCR5+ and U87-CD4+ CXCR4+, respectively) were donated by Dan Littman (Howard Hughes Medical Institute, New York, N.Y.). They were maintained in Dulbeccos minimal essential medium (Sigma) containing 10% FBS, glutamine, penicillin/streptomycin, puromycin (1 g/ml, Sigma) and geneticin (G418; 300 g/ml, Sigma), and were split twice a week, as described [15].

Virus isolates and serum samples: The following HIV-1 isolates were used: (1) Ba-L, 168.1, 168.10 and T-CSF (donated by Dr. Michael A. Norcross, CBER, US FDA, Bethesda, Md.); (2) 95BRRJ10, 95BRSP01, 95BRSP07 and 95BRBA07, which were isolated in our laboratory, as described [5]. Stock viruses have been kept at −70° C., and expanded only in PBMCs from HIV-1-seronegative blood donors, except T-CSF, which has been expanded in the CD4+ tumor cell line PM-1. The general phenotypic characteristics and the preferential coreceptor usage of the isolates have already been reported by us [1,1]. In summary, Ba-L and 168.1 are macrophage-tropic, non-syncytium-inducing (NSI) and R5X4-using viruses; the TCLA virus T-CSF and the primary isolates 95BRRJ10 and 95BRSP01 are X4-tropic, syncytium-inducing (SI) variants; 95BRBA07, 95BRSP07 and 168.10 are R5X4-using, SI isolates. Serum samples from HIV-1-positive individuals and from normal donors were provided by the Brazilian Network for HIV Isolation and Characterization [5], inactivated at 56° C. for 30 min, and stored at −70° C. until use.

Monoclonal antibodies: The anti-S. mansoni carbohydrate mAbs E1, E3 and E5 were obtained in BALB/C mice immunized with either egg or soluble egg antigens of S. mansoni, as previously described [17]. mAbs E1 (IgG2b) and E3 (IgG3) recognize oligosaccharide epitopes containing fucose on their structure. mAb E5 (IgM) reacts with the oligosaccharide lacto-N-fucopentaose III (LNFPIII), which contains the Lewis X sugar on its structure. E1 and E5 were purified by protein A or anti-IgM chromatography, then dialyzed against PBS. E3 was salted out of culture supernatant, and then also dialyzed against PBS. The mAbs were filtered before using in the neutralization studies.

Viral neutralization by human sera HIV-1-positive supernatants (5 ng/ml p24 Ag) were incubated with HIV-1 antibody-positive human serum (RJ31 or SP09), at a final dilution of 1:100, for 60 min at 37° C., and the virus-serum suspension was added to transfected U87 cells previously seeded in 96-well flat-bottom culture plates (1×104/well). After overnight incubation, cells were washed, fresh medium was added back and culture was maintained at 37° C., 5% CO₂, for 7-10 days. Viral replication was evaluated by detecting the activity of the enzyme reverse transcriptase (RT) in culture supernatants, as described [16]. HIV-1 antibody-negative human serum was used as a control. Neutralization of R5- and X4-tropic isolates was studied infecting U87-CD4+CCR5+ or U87-CD4+CXCR4+ cells, respectively, and of R5X4-tropic isolates infecting both cells.

Virus neutralization by anti-carbohydrate mAbs: HIV-1-positive cell-free supernatants (5 ng/mal p24 Ag) were incubated with different concentrations of mAbs and, after 1 h at 37° C., the mAb-virus mixture was added to PHA-activated PBMCs in 96-well flat-bottom culture plates (2×105/well per 2001). Cultures were incubated overnight at 37° C., 5% CO2, and cells were washed to remove the excess of virus and antibodies. Regular medium with 5 U/ml IL-2 was added back, and cells were cultured for additional 7-10 days. The same procedures were done with irrelevant mAbs as a control, and virus replication was assessed by detecting the RT activity in the culture supernatants [16].

Results. Before addressing the sensitivity of the primary isolates to HIV-1 antibody-positive serum samples or to anti-S. mansoni carbohydrate mAbs, we selected human sera presenting a potent neutralizing activity against the HIV-1 TCLA isolate T-CSF, which has been shown to be highly sensitive to antibody neutralization [4]. Several randomly chosen serum samples were tested, and all of them readily neutralized this variant (data not shown). Two of these sera (RJ31 and SP09) were eventually selected according to their ability to inhibit at least 90% of T-CSF replication in U87CD4+CXCR4+ cells at dilutions not under 1:100.

Figure taken from the experiments shows HIV-1 sensitivity to human sera. U87-transfected cells were exposed to viral supernatants preincubated or not with HIV-1 antibody-positive human serum (dilution 1:100). Cells were washed, fresh medium was added back and culture was maintained for 7-10 days. Viral replication was evaluated by detecting the RT activity in culture supernatants, and data represent the means ±SEM of four experiments done in triplicates. R5 and X4 indicate the cells U87-CD4+CCR5+ and U87-CD4+CXCR4+, respectively. HIV-1 antibody-negative human serum did not affect viral replication (RT reverse transcriptase).

Subsequently, we analyzed the sensitivity of five primary isolates of HIV-1 to serum samples RJ31 and SP09, in the context of coreceptor usage. We showed that the isolates Ba-L (R5-tropic), 95BRRJ10 (X4-tropic) and 95BRBA07 (X4R5-tropic) were completely resistant to both sera at 1:100 dilution, whereas the isolate 95BRSP01 (X4-tropic) was mildly inhibited by serum SP09. The dual-tropic isolate 95BRSP07 was only moderately inhibited by serum RJ31 in U87CD4+CCR5+ cells, but it was completely resistant to this serum in infection assays using U87CD4+CXCR4+ cells (virus 95BRSP07 was not tested with serum SP09).

We next examined the possible neutralizing activity of anti-carbohydrate mAbs against primary isolates of HIV-1 presenting different tropisms for chemokine receptors. In marked contrast with the observed resistance to neutralization by HIV-1 antibody-positive serum samples, the replication of HIV-1 isolates was inhibited by anti-carbohydrate mAbs in a concentration-dependent manner, irrespective of the virus preferential coreceptor usage. Regarding the R5-tropic isolates (FIG. 2), virus Ba-L was highly sensitive to mAbs E1 and E3, with inhibition of infection ranging from 60% to 83% with 10 g/ml to 40 g/ml of each mAb, and partially blocked by E5 (54% inhibition with 40 g/ml). The isolate 168.1 was resistant to mAbs E1 and E5, but it was inhibited (66%) by 40 g/ml of E3. Concerning the neutralization of X4 isolates, virus 95BRRJ10 was moderately (55%) to strongly (78%) inhibited by 20 g/ml and 40 g/ml of mAbs E3 and E5. mAb E1 showed weak or no blocking activity against the X4 virus samples.

Relative to R5X4-tropic viruses, we found that all mAbs neutralized the isolates 95BRSP07 and 95BRBA07 at 40 g/ml, with levels of inhibition ranging from 50% to 73% and from 50% to 67%, respectively. Isolate 168.10 evaded the neutralizing activity of the mAbs E1 and E5, but it was sensitive to 20 g/ml (52% inhibition) and 40 g/ml (66% inhibition) of E3.

Figure taken from these experiments showed inhibition of R5-tropic isolates by anti-carbohydrate mAbs. Peripheral blood mononuclear cells were exposed to viral supernatants preincubated or not with different concentrations of mAbs. Cells were washed, regular medium with 5 U/ml IL-2 was added back, and culture was maintained for 7-10 days. Viral replication was evaluated by detecting the RT activity in culture supernatants, and data represent the means ±SEM of four experiments done in triplicates.

A figure taken from these experiments also showed inhibition of X4-tropic isolates by anti-carbohydrate mAbs. Figure showed inhibition of X4-tropic isolates by anti-carbohydrate mAbs. Peripheral blood mononuclear cells were exposed to viral supernatants preincubated or not with different concentrations of mAbs. Cells were washed, regular medium with 5 U/ml IL-2 was added back, and culture was maintained for 7-10 days. Viral replication was evaluated by detecting the RT activity in culture supernatants, and data represent the means ±SEM of four experiments done in triplicates. Abbreviations following the virus names indicate the mAb used. Irrelevant control mAbs did not affect viral replication Virus denominations were shortened for simplification, and abbreviations following the virus names indicate the mAb used.

Figure taken from these experiments showed inhibition of R5X4-tropic isolates by anti-carbohydrate mAbs. Peripheral blood mononuclear cells were exposed to viral supernatants preincubated or not with different concentrations of mAbs. Cells were washed, regular medium with 5 U/ml IL-2 was added back, and culture was maintained for 7-10 days. Viral replication was evaluated by detecting the RT activity in culture supernatants, and data represent the means ±SEM of four experiments done in triplicates.

Discussion. Primary isolates of HIV-1 are remarkably resistant to antibody neutralization, either to hyperimmune sera from vaccines or from HIV-1-infected individuals [9]. The antibody resistance exhibited by primary isolates is related to reduced exposure of immunodominant epitopes in the glycoprotein (gp120/41) of the viral envelope [4, 25, 32]. In this work, we also observed that immune sera of HIV-1-infected individuals could not inhibit the infection of some HIV-1 primary isolates. In agreement with other authors [7, 18, 21, 27], we did not find clear evidence that the preference for using the coreceptors CXCR4 or CCR5 was a critical property determining the level of sensitivity of primary isolates of HIV-1 to neutralization by anti-HIV-1 serum samples.

Carbohydrates constitute approximately 50% of the gp120/41 mass [19] and may hide antigenic peptide epitopes from antibodies, limiting the protective efficiency of the humoral immune response against HIV-1 [23, 32]. On the other hand, the glycan residues of the viral envelope can function as neutralization sites [13, 14, 24, 26]. In this study, we report that anti-carbohydrate mAbs, raised against the egg antigen of S. mansoni, could neutralize seven primary isolates of HIV-1, and that the inhibitory activity occurred irrespective of the preferential coreceptor usage of the isolates.

The anti-glycan mAbs clearly inhibited the CCR5-using isolates Ba-L and 168.1 (FIG. 2), the CXCR4-tropic isolates 95BRRJ10 and 95BRSP01 (FIG. 3) and the dual-tropic, CCR5/CXCR4-using viruses 168.10, 95BRSP07 and 95BRBA07 (FIG. 4). The neutralization of these isolates was consistent and reproducible, despite minor variations in the intensity of the inhibition mediated by each mAb, implying that putative antibodies induced by gp120/41 carbohydrate residues may be very effective against HIV-1. Importantly, five isolates (Ba-L, 95BRRJ10, 95BRSP01, 95BRBA07 and 95BRSP07) displayed a marked resistance to human sera endowed with potent anti-HIV-1 activity.

We have examined whether these anti-glycan mAbs bind to human PBMCs, by FACScan and by Western blot, and we found no binding (preliminary results, not shown). This suggests that the anti-HIV-1 activity may be due to direct binding to the virus, albeit we have not yet performed the binding assays on the virus. It is possible that the mAbs recognize epitopes located near to either CD4 or coreceptor binding sites on the viral envelope. Reactions between the mAbs and the ubiquitous carbohydrate determinants at key locations on gp120/41 could prevent the virion-cell membrane interaction and fusion by steric hindrance, forming a nonspecific blockade to infection. Hansen et al. [13] identified four carbohydrate epitopes on HIV-1 envelope that are recognized by neutralizing antibodies, and two of them, Lewisy (also found in S. mansoni egg antigen) and A, have fucose on their structure. We could envisage that the mAbs E1, E3 and E5, which recognize fucose-containing epitopes, inhibit HIV-1 by binding to those determinants and, possibly, to other similar glycan residues on gp120/41.

Our findings indicate that the carbohydrates of the HIV-1 viral envelope may be a target for an effective humoral immune response elicited by vaccination. In fact, rabbits immunized with the mucin-type carbohydrate sialosyl-Tn, which is present on the HIV-1 gp120/41 [14], developed neutralizing antibodies against the laboratory-adapted isolates IIIB and MN [8]. Moreover, the broadly neutralizing human anti-HIV-1 mAb 2G12 recognizes a mannose-dependent epitope, composed primarily of carbohydrates, with no involvement of gp120 peptides [24,26]. In conclusion, our study contributes to the knowledge concerning the development of reliable immunogens able to elicit a protective humoral immunity against HIV-1.

Further studies should address other relevant aspects, such as a comparative analysis of neutralizing potency against different HIV-1 subtypes. Curiously, recent reports have described that patients infected with HIV-1 and with acute infections by Orientia tsutsugamuchi [30], measles [22] or dengue viruses [31] presented a significant decline in the plasma HIV-1 load, and in vitro studies showed that the human herpes virus-6 suppressed HIV-1 replication in lymphoid tissues by inducing the production of -chemokines [12]. These clinical and experimental findings, together with our present results, may reveal the other side of the coin of the association between HIV-1 and co-pathogens.

### REFERENCES

1. Adachi M, et al., (1988) J Exp Med 167:323-331
2. Berger E A, et al., (1999) Annu Rev Immunol 17:657-700
3. Borrow P., et al., (1994) J Virol 68:6103-6110
4. Bou-Habib D C, et al., (1994) J Virol 68:6006-6013.
5. Brazilian Network for HIV Isolation and Characterization (2000) HIV-1 diversity in Brazil: genetic, biologic, and immunologic characterization of HIV-1 strains in three potential HIV vaccine evaluation sites. Brazilian Network for HIV Isolation and Characterization. J Acquir Immune Defic Syndr 23:184-193
6. Burton R, Moore J P (1998) Nat Med 4 (Suppl 5):495-498
7. Cecilia D, et al., (1998) J Virol 72:6988-6996.
8. Clausen H, Sorensen T, White T, Wandall H H, Hansen J E S (1994) Simple mucin type O-glycans of HIV: enzymatic prediction of glycosilation sites for vaccine construction. In: Bock K, Clausen H, Krogsagaard-Larsen P, Kofod H (eds) Complex carbohydrates in drug research. Alfred Benzon Symposium, Copenhagen, pp 414-427
9. Cohen J (1993) Jitters jeopardize AIDS vaccine trials. Science 262:980-981
10. Cohen O J, Weissman D, Fauci A S (1999) The immunopathogenesis of HIV infection. In: Paul W E (ed) Fundamental Immunology. Lippincott-Raven, Philadelphia, pp 1455:1509.
11. Ferraro G A, Mello M A, Sutmoller F, Van Weyenbergh J, Brazilian Network for HIV Isolation and Characterization, Shindo N, Galvao-Castro B, Bou-Habib D C (2001) AIDS Res Hum Retroviruses 17:1241-1247
12. Grivel J C, et al., (2001) Nat Med 7:1232-1235.
13. Hansen et al., (1990) J Virol 64:2833-2840.
14. Hansen J E, et al., (1991) J Virol 65:6461-6467.
15. Hill C M, et al., (1997) J Virol 71:6296-6304
16. Hoffman A D, Banapour B, Levy J A (1985) Virology 147:326-335.
17. Ko A I, et al., (1990) Proc Natl Acad Sci USA 87:4159-4163.
18. LaCasse R A, et al., (1998) J Virol 72:2491-2495.
19. Leonard C K, et al., (1990) J Biol Chem 265:10373-10382.
20. Migueles S A, et al., (2002) Nat Immunol 3:1061-1068.
21. Montefiori D C, et al., (1998) J Virol 72:1886-1893.
22. Moss W J, et al., (2002) J Infect Dis 185:1035-1042.
23. Reitter J N, Means R E, Desrosiers R C (1998) Nat Med 4:679-684
24. Sanders R W, et al., (2002) J Virol 76:7293-7305.
25. Sattentau Q J, Moore J P (1995) J Exp Med 182:185-196.
26. Scanlan C N, et al., (2002) J Virol 76:7306-7321.
27. Trkola A, et al., (1998) J Virol 72:1876-1885
28. Ugolini S, Mondor I, Sattentau Q J (1999) Trends Microbiol 7:144-149.
29. Velupillai P, Ham D A (1994) Proc Natl Acad Sci USA 91:18-22.
30. Watt G, et al., (2000) Lancet 356:475-479.
31. Watt G, Kantipong P, Jongsakul K (2003) Clin Infect Dis 36:1067-1069.

32. Wei X, Decker J M, et al. (2003) Nature 422:307-312

33. Zhu T, et al. (1993) Science 261:1179-1181.

### EXAMPLE 2

We showed that the levels of viral p24 as determined by ELISA. As can be seen in Table 1, all three Mabs had neutralizing activity against primary isolates of HIV-1. In general, highest levels of neutralization for any Mab were between 70-80% suppression of viral p24 levels compared to controls. We performed these experiments with multiple doses of Mab and in general obtained the highest levels of neutralization at Mab concentrations of 20-40 ug/ml (Table 1).

TABLE 1
Inhibition of HIV-1 replication by anti-glycan E.1, E.3 and E.5
monoclonal antibodies.

| | Isolate SP95/001^{a} | | | Isolate SP95/007^{a} | | |
|---|---|---|---|---|---|---|
| | Monoclonal | | | Monoclonal | | |
| Concentration | Antibody | | | Antibody | | |
| (μg/ml) | E.1 | E.3 | E.5 | E.1 | E.3 | E.5 |
| 40 | 18.20^{b} | 74.59 | 76.00 | 28.56^{b} | 56.93 | 72.62 |
| 20 | 26.71 | 79.70 | 59.64 | 21.18 | 48.33 | 52.94 |
| 10 | 00.60 | 26.48 | 18.81 | 13.08 | 51.85 | 00.63 |

^{a}T cell Tropic.
^{b}Percentage of neutralization (based on P24 levels determined by ELISA at day 7 in culture) compared to pooled isotype matched monoclonal antibodies.

Additional data from neutralization experiments against additional primary isolates of HIV-1 from Brazil was also produced. In all cases, control, isotype matched mabs did not neutralize primary isolates of HIV-1 at any concentration tested. In addition, these data show that each of these mabs is able to neutralize M-tropic, T-tropic and dual-tropic virus. Further, each of the mabs vary in their ability to strongly neutralize the various primary isolates of HIV-1 they were tested against, supporting our hypothesis that a multiple glycan epitope vaccine will be needed to induce broadly neutralizing abs. For most of the primary isolates, E.3 demonstrates the strongest neutralizing activity, with other isolates such as RJ10, mab E.5 neutralizes as well as E.3 and for primary isolate BA07, E.5 exhibits the strongest neutralizing activity. E.1, never exhibits the strongest neutralizing activity, usually falling somewhere between E.3 and E.5 for most of the isolates tested against. We believe that these data strongly suggest that LNFPIII, or Lewis X, or a conformational mimic will be found on HIV-1 envelope and likely the glycan structures recognized by Mabs E1 and E3. Further evidence that the glycans recognized by E.1, E.3, and E.5 are found on gp120 was also detected.

As shown in the Table 1, our three anti-glycan mabs are each able to neutralize a variety of primary isolates of HIV-1 from Brazil. The glycan target of mab E.5 contains the asialo, asulfo Lewis X trisaccharide, which was shown to bind to DC-SIGN and inhibit HIV-1 gp120 binding to DC-SIGN. Based on this observation we felt it was likely that E.5 would bind to HIV-1 gp120. We screened the mAbs for binding to native gp120 obtained from M-tropic JRFL strain of HIV-1 by ELISA, comparing binding of our mabs with that of the neutralizing mab 2G12, which recognizes an oligo-mannose dependent epitope which does not bind to DC-SIGN. As shown in FIG. 6, we found that the mAbs bound to gp120, with E.1 and E.5 binding fairly strongly. We showed that E-series mAbs are able to neutralize the R5X4-tropic syncitium inducing HIV-1 primary isolate SP07. We also showed that E-series mAbs are able to neutralize the lab adapted R5 macrophage-tropic non-syncitium inducing strain Ba-L, and that shows E-series mAbs are able to neutralize the X4-tropic syncitium inducing HIV-1 primary isolate RJ10

Targeting vaccines to dendritic cells using Heat Shock Protein constructs. We genetically fused the extracellular hydrophilic loop of our schistosome DNA vaccine Sm23 at its N- or C-terminus to murine Hsp70 (accession #. M76613). In addition, we cloned the different combinations in the prokaryotic expression plasmid pTrc-HisB (Invitrogen) to produce the different recombinant his-fusion proteins, and this is the approach that we propose to use to produce TT-Hsp 70 fusion carrier protein for the proposed experiments. Recombinant TT-Hsp70 proteins will be purified using Nickel-Chromatography as previously described (Da'dara AA, et al., Vaccine 2001; 20: 359-369). We tested for production of recombinant Hsp70, Sm23-Hsp70 and Hsp70-Sm in E. coli. IPTG-induced bacterial cells containing the different expression plasmids were lysed, purified using nickel chromatography and separated onto SDS-PAGE. An SDS-PAGE showed the expression and purification of Sm23/Hsp70. The different proteins were expressed in E. coli using pTrcHis plasmid and purified using Nickel chromatography. Hsp70, Sm23-Hsp70, and Hsp70-Sm23. we used Kaleidoscope marker.

Addition of Hsp70 to a schistosome antigen significantly enhances antibody responses. We tested whether adding the DC targeting Hsp70 fragment to our schistosome vaccine candidate Sm23 would lead to enhanced immune responses compared to vaccinating mice with recombinant Sm23 alone. We showed that vaccination with the Hsp70-Sm23 fusion protein led to a large and significant increase in the amount of IgG2a and IgG2b antibodies to Sm23 compared to mice immunized with Sm23 alone. Mice were immunized with recombinant Sm23 alone or with the fusion Hsp70 (N-terminal)/Sm23 (Middle bar) or with Hsp 70 (C-terminal)/Sm23 (Right Bar) and boosted in an identical manner 4 weeks later. We showed the IgG2a anti-Sm23 response, and the IgG2b anti-Sm23 antibody response. These data supports our rationale and hypothesis that Hsp70 does enhance immune responses, notably antibody responses, and should function to enhance the response to LNFPIII, E.1 and E.3 conjugate vaccines.

Hsp70 enhances immune response to HIV-1 epitope p18. We compared immunogenicity of his TD158 multi-epitope CTL plasmid DNA vaccine plus or minus Hsp70. This vaccine contains a 15 amino acid (P18; RIQRGPGRAFVTIGK (SEQ ID NO: 1)) immunodominant peptide of HIV-1 gp120 recognized by CD8+ CTL with the class I molecule H2D^{d}. To evaluate the effect of Hsp70 on the CTL immune response to HIV-1 vaccine, we first cloned the murine Hsp70 coding region at the amino or carboxyl end of P18 CTL epitope vaccines. To enhance secretion of the vaccine antigen, we also produced constructs containing the murine Ig-k leader sequence.

We showed that immunization with the secreted form P18-Hsp70 resulted in a significant increase in the CD8+ immune to P18 as determined by ELISPOT.

Production of LNFPIII conjugates. The experiments described in Objectives 1 and 3 rely on the production of LNFPIII-TT conjugates and LNFPIII-TT-Hsp70 conjugates respectively. To date we have had LNFPIII conjugates prepared using dextran, human serum albumin and bovine serum albumin as carrier molecules, by Neose Technologies Inc., and by Dr. Thomas Norberg of Sweden. We were provided with approximately 1 gram of LNFPIII by Neose Technologies and this is the oligosaccharide we will use to produce the TT and TT-Hsp70 conjugates described in Activities 1 and 4. FIGS. 10A and 10B show the structure of LNFPIII as well as the nature of the conjugate, using dextran as the carrier molecule. FIG. 9 is an HPLC of the pure LNFPIII.

Production and purification of recombinant TT and TT-Hsp70. For the TT-Hsp 70 fusion protein we will produce recombinant tetanus toxin C fragment The pTrcHisB plasmid construct will be used to introduce the tetanus toxin C fragment in this plasmid. The fragment C of the tetanus toxin (TT) gene will be amplified by PCR using the following primers (designed based on a data base sequence with accession number X04436): 5′-end primer: GGCCATGGGGCATCATCATCATCATCATAAAGATTC-CGCGGGAGAAG (SEQ ID NO:2), with NcoI site underlined and 6 histidines, italic; and the 3′-end primer: GGCTCGAGATCATTTGTCCATCCTTCATC (SEQ ID NO:3) with XhoI site, 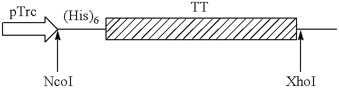
underlined. The amplified product will be cloned in the Hsp-Trc plasmid as described. The amplified products will be digested with NcoI and XhoI, purified and cloned in the NcoI/XhoI pre-digested pTrc plasmid. This will result in the production of the construct TT (see Figure below). The construct will be sequenced and introduced into AD494(DE3) E. coli bacterial strain. The expression of the fusion protein will be induced by 1 mM IPTG. The recombinant fusion protein will be purified using affinity Nickel chromatography using standard procedures. In order to remove the LPS contamination, the recombinant fusion protein will be further purified on endotoxin removal column (Detoxi-Gel endotoxin removing gel, Pierce, Rockford, Ill.) according to manufacturer's instructions.

Briefly, we will prepare purified recombinant TT in an identical manner to that described in Preliminary Studies for Hsp 70, using a nickel column, 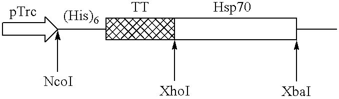
and then removal of bacterial endotoxin using a polymixin column. We will genetically fuse TT to the N-terminus of murine Hsp70 (accession #. M76613, see Figure below). The TT-Hsp 70 combinations will be cloned in the prokaryotic expression plasmid pTrc-HisB (Invitrogen), to produce recombinant proteins as His-fusion proteins, which we will purify using Nickel-Chromatography as previously described (Da'dara AA, et al., Vaccine 2001; 20: 359-369).

As an alternative to modern linker-spacer conjugation methods we can also use standard methods as described (Paoletti L C and Kennedy R C, J Infect Dis 2002; 186: 1597-1602; Steinhoff M and Goldblatt D, Lancet 2003; 361: 360-361; Curran M P, et al., Drugs 2003; 63: 673-682; discussion 683-674; Ada G and Isaacs D, Clin Microbiol Infect 2003; 9: 79-85; Obaro S K, Clin Microbiol Infect 2002; 8: 623-633; Rice J, et al., J Immunol 2001; 167: 1558-1565; Lee J J, et al., Infect Immun 2000; 68: 2503-2512; Stratford R, et al., Vaccine 2001; 20: 516-525; Cheng Q, et al., Infect Immun 2002; 70: 6409-6415), conjugating LNFPIII directly to TT using mild sodium meta-periodate oxidation of the pentasaccharide, or the pentasaccharide conjugated to dextran as described in Paoletti and Kennedy (J Infect Dis 2002; 186: 1597-1602). To create TT-LNFPIII or TT-Hsp70-LNFPIII conjugates, the periodate treated LNFPIII are mixed with carrier at a 2:1 ratio of sugar to protein in a saline citrate buffer pH 6.0. Conjugation is started by addition of sodium cyanoborohydride with reactions proceeding for approximately 14 days at 37° C. with additional sodium cyanoborohydride added on days 7 and 12 essentially as described in Paoletti and Kennedy (J Infect Dis 2002; 186: 1597-1602). The problem with the latter method is that many of the glycans will be “flat” on to the carrier and not presented to immune cells as a separate entity. Using linker-spacer technology allows for three-dimensional rotation on axis of the glycan away from the carriers, and better presentation to immune cells.

### EXAMPLE 3

Previously, we reported that established cell mediated immunity waned as CMS5 tumors progress. T cells from tumor-bearing mice developed signaling defects and made weak Th1-dependent responses to flu. Moreover, spleens of these mice contain increased numbers of Gr1+/CD11b+myeloid cells that suppressed T cell function. In another series of experiments, we showed that levels of Gr1+/CD11b+ cells also were elevated in mice with schistosomiasis. These cells, induced by Lewis X-containing glycans on schistosome eggs, suppressed Th1 responses and promoted Th2 responses, fostering host survival. Here we report that CMS5 tumor cells express Lewis X-containing glycans and that, as in schistosomiasis, the increase in Gr1+/CD11b+ cells in tumor-bearing mice is associated with decreased Th1 responses and increased Th2 responses. The findings suggest the hypothesis that Lewis X-containing glycans on CMS5 modulate immunity, but, contrary to schistosomiasis, enhance the development of deleterious Th2 responses and the loss of protective Th1 responses, favoring tumor survival.

Introduction:

Our understanding of the development of immune mechanisms effective at destroying tumor cells has deepened during the past several years, providing a framework within which to study the failure of tumor rejection (17;18). Minimally, an effective response requires target antigens on the tumor cells, effector precursors capable of recognizing them, and regulatory cells that foster the development of appropriate effector responses. Several hypotheses have been advanced to explain why the system often fails. These include poor immunogenicity of tumor associated antigens (TAA) (1,2), the weak ability of solid tumor cells to deliver costimulatory signals (19), the action of cytokines and growth factors, such as VEGF, IL-10 and TGF, that suppress effector cell maturation and functional ability, and the ability of regulatory T cells (Treg) to suppress the development of responses against the tumor (4,5). However, reports that tumor immunity that develops initially is lost before the tumor is destroyed, suggested that mechanisms exist to suppress tumor immunity even after it develops. Supporting evidence for this hypothesis was provided by Ghosh et al. who showed that early Th1 responses against the MCA38 tumor decreased in late tumor-bearing mice (20). In addition, Watson et al. found that immunity against mammary carcinoma declined to pre-sensitization levels within the first month (21). Finally, Fu et al. demonstrated that T cells from mice with advanced tumors lost the ability to reject metastases seen with T cells from mice with smaller tumors (8).

Previously, we reported that intradermal injection of CMS5 cells, a weakly immunogenic fibrosarcoma, induced an early anti-tumor response in parental tumor-bearing mice that waned with time (9). Cytotoxic T lymphocytes (CTL) generated from spleen cells of mice injected 9-10 days earlier with tumor cells lysed CMS5 cells in an antigenically specific fashion. However, the tumors continued to increase in size and by day 28 cytotoxicity was no longer detectable. In addition, the mice had very low levels of the TCR^{˜} chain and p56lck, proteins involved in signal transduction, and lost the ability they had initially to generate anti-viral CTL, a Th1-dependent response (13;14). Mice that lost anti-tumor immunity exhibited massive splenomegaly due to the infiltration of Gr1+/CD11b+myeloid cells (5). Several groups have described tumor and virus models in which Gr1+/CD11b+ cells suppressed T cell activation and/or function via mechanisms that involved reactive oxygen species, nitric oxide, and arginase (12-17). Following tumor resection, the level of Gr1+/CD11b+ cells returned to normal and, consistent with a role for these cells in suppressing immunity, anti-tumor immunity returned by 24-48 hours (5).

In schistosome-infected mice strong Th1-dependent responses develop initially, but decrease with time and are replaced by dominant Th2 responses (18). Recently, we reported that the inoculation of a conjugate of dextran or human serum albumin (HSA) and glycans containing the Lewis X trisaccharide, found on schistosome eggs, induced Gr1+/CD11b+ cells and acted as a Th2 adjuvant (19-21). These results are consistent with those from several groups showing that molecules expressed (22-25) by helminthes play an important role in regulating immunity to the worms. Of particular interest, given our results, was the report by McKee and Pearce that IL-10-secreting CD4+/CD25+ Treg cells in schistosome-infected mice inhibited IL-12, suppressing Th1 responses and enhancing Th2 responses (26). In the present study we report that CMS5 cells share glycans containing the Lewis X-trisaccharide with schistosome eggs and that, as the tumor progresses, Th1 responses decrease and Th2 responses increase. Moreover, depletion of CD4+/CD25+ cells in naïve mice, before the injection of tumor cells, prevents tumor growth. Based upon our findings, we hypothesize that Lewis X-containing glycans on CMS5 cells induce Gr1+/CD11b+ cells, leading to the development of deleterious Th2 responses and promoting the loss of the protective Th1 responses, thereby favoring survival of the tumor.

Materials and Methods:

Mice and tumor cells: Female BALB/c mice, 6-8 weeks of age, were obtained from Charles River (Wilmington, Mass.). CMS5, a methylcholanthrene-induced fibrosarcoma of BALB/c origin, MCA38, a murine colon carcinoma, Lewis lung carcinoma, and B16 melanoma cell lines were used for these experiments. All cell lines were from the American Type Culture Collection (Manassas, Va.). CMS5 and B16 cell lines were grown in DMEM plus 10% FCS (Hyclone, Logan, Utah), supplemented with 100 U/ml penicillin, 100 ug/ml streptomycin, and 2 mM glutamine. MCA38 and Lewis Lung cell lines were grown in RPMI 1640 plus 10% FCS supplemented as above. Media and supplements were obtained from Gibco (Grand Island, N.Y.).

For in vivo inoculation, 0.5×106 CMS5 cells were injected subcutaneously (s.c.) in the back of 3-5 mice. Tumor growth was measured using Vernier calipers and reported as mm2 as the product of “a”×“b”, where “a” is the (longest surface length) and “b” is the longest surface width. Following the inoculation of tumor cells, spleen cells were harvested from early tumor-bearing mice after 9-10 days and from late tumor-bearing mice after 28 days.

Flow cytometry: To identify Gr1+/CD11b+ cells, spleen cells were pooled from mice (five/group/data point) and reacted with 100 ng of anti-Gr1+-FITC (RB6-8C5, rat IgG2b) or anti-CD11b-PE (M1/70, rat IgG2b) both from BD Pharmingen, San Diego, Calif. Unlabeled, purified anti-Gr1+ antibody was prepared by growing the RB6-8C5-producing hybridoma in AIM 5 media (Gibco). Antibodies were purified by the Mount Sinai Hybridoma Core Facility using protein G sepharose columns. Anti-CD3ε-FITC (145-2C11, IgG1, BD Pharmingen) was used to determine the percentages of mature T cells. Cells (1×10⁶) were reacted with the desired antibody for 30 minutes at 4° C. Irrelevant isotype matched antibodies were used as controls. Samples were washed twice in FACS buffer (PBS with 0.1% BSA and 0.01% sodium azide). Flow cytometric acquisition and analysis was performed on a FACScan cytometer running CellQuestPro software (BD, San Jose, Calif.),

The mouse antibody, E.5 (IgM), prepared by immunizing mice with S. mansoni eggs, recognizes an asialo, asulfo Lewis X sugar, was prepared as described (27). After incubation of 1 ug of E.5 or isotype control antibody/106 cells, cells were washed, mixed with goat anti-mouse IgM-FITC (Biomeda Corp, Foster City, Calif.), washed again, and analyzed by FACS as above.

Induction of Th1 Responses to Influenza Virus and Th2 Responses to Keyhole Limpet Hemocyanin (KLH):

Influenza virus A/PR8/8/34 (PR8) was used to induce a Th1 response. PR8 (kindly provided by Dr. Thomas Moran, Mount Sinai School of Medicine) was grown in the allantoic cavity of embryonated hen eggs and stored at −70° C. Viruses were titered by determining infectivity of MDCK cells and expressed as tissue culture infectious units (TCIU) (28). Naïve, early tumor-bearing and late tumor-bearing mice (3-5 per group) were immunized with PR8. After 7 days mice were sacrificed and single cell suspensions of splenocytes, devoid of erythrocytes, were prepared for use as responder cells. APC were x-irradiated splenocytes (1800 rads) from naïve mice, prepared as for responders. They were infected with 5×106 TCIU of PR8 for 1 h at 37° C. and washed. Responder cells, 1×106/ml, with or without APC, also at 1×106/ml, were cultured in RPMI, supplemented with 10% FCS (Hyclone), 100 U/ml penicillin, 100 ug/ml streptomycin, 2 mM L-glutamine, 0.1 mM NEAA, 1 mM sodium pyruvate and 5×10−5M 2ME (Gibco). After 72 h supernatants were harvested and analyzed for levels of IFN□ using ELISA test kits as per the manufacture's instructions (R&D Systems, Minneapolis, Minn.).

To induce a Th2 response mice (3-5 per group) were primed with 80 ug KLH i.p. (Calbiochem, La Jolla, Calif.). After 9 days, splenocytes were prepared as above and cultured at 1×106/ml with or without KLH. Culture supernatants were assayed for levels of IL-4 and IL-10 using ELISA test kits as per the manufacture's instructions (R&D Systems, Minneapolis, Minn.).

In some experiments APC, enriched or depleted for Gr1+CD11b⁺, were prepared by incubating splenocytes with purified anti-Gr1 antibody at 1 ug/106 cells, washed, incubated with magnetic MicroBeads labeled with anti-rat antibody and purified on Miltenyi columns as per the manufacture's instructions (Miltenyi, Biotec, Auburn, Calif.). Bound cells represented the enriched fraction and the flow-through represented the depleted fraction, respectively.

Results:

CMS5 Cells Express Lewis X-Containing Glycans.

Previously, we reported that the growth of CMS5 tumors in vivo led to the loss of established Th1-dependent responses to flu virus (11). Numbers of Gr1+/CD11b+ cells in early tumor-bearing mice that made good anti-tumor and anti-viral responses resembled those found in naïve mice. In contrast, late tumor-bearing mice that lost immunity contained increased numbers of Gr1+/CD11b+ cells, shown in several systems to suppress T cells (12-17). We showed that levels of G1⁺/CD11b+dual staining cells are increased in late tumor-bearing mice. Mice were injected with 0.5×10⁶ CMS5 cells on day −28 (late tumor bearing) or day −9 (early tumor bearing) prior to sacrifice. Splenocytes were stained with 100 ng of anti-Gr1-FITC and 100 ng of anti-CD11b-PE, as described in the Materials and Methods section. Total splenocytes were analyzed via flow cytometry. Similar results were obtained in 6 experiments.

In a separate series of experiments, we observed increased levels of Gr1+/CD11b+ cells in mice with schistosomiasis and showed that these cells could be induced by inoculating conjugates of dextran or HSA and purified Lewis X trisaccharide-containing glycans, found on schistosome eggs (19,20). During the natural course of schistosomiasis, Th1 responses decrease at about week 4, at which time Th2 responses become dominant (18). Studies we reported showed that Lewis X trisaccharide-containing glycans promoted the development of Th2 responses (21). It is well documented that carbohydrate antigens, including Lewis X-containing glycans, also are expressed on solid tumor cells where they have been shown to play a role in tumor metastasis (29). Since the increase in Gr1+/CD11b+ cells and the Th1/Th2 switch was induced by inoculating conjugates of dextran or HSA and purified Lewis X trisaccharide-containing glycans, and since certain tumor cells express these ligands, we hypothesized that Lewis X-containing glycans regulated the loss of immunity in tumor-bearing mice by inducing Gr1+/CD11b+ cells and the subsequent loss of Th1-dependent responses.

To test for the expression of these molecules, tumor cells were subcultured and the binding of the monoclonal antibody E.5, directed against Lewis X glycans on schistosome eggs, examined at 24 and 48 hours. Results were analyzed by flow cytometry. E.5 antibodies bound to CMS5 and B16 cells by 24 hours. It bound to MCA38 and Lewis Lung cells also, although not until 48 hours, consistent with the molecules being regulated and not constitutively expressed. We showed that levels of G1+/CD11b+dual staining cells are increased in late tumor-bearing mice. Mice were injected with 0.5×106 CMS5 cells on day −28 (late tumor bearing) or day −9 (early tumor bearing) prior to sacrifice. Splenocytes were stained with 100 ng of anti-Gr1-FITC and 100 ng of anti-CD11b-PE, as described in the Materials and Methods section. Total splenocytes were analyzed via flow cytometry. Similar results were obtained in 6 experiments Similar results were obtained in 4 experiments.

As CMS5 Tumors Grow, the Th1 Response Diminishes, while the Th2 Response Increases.

The immune response to helminth parasites that enables clearance of the worn is characterized by an early Th1 response that is replaced by a Th2 response after several weeks (18). The Th2 response is generally considered protective, since it reduces the initial inflammation and also leads to the generation of IgE antibodies. In fact, IL-4−/− mice die of the infection (30). A variety of glycans found on parasites, including schistosomes, have been shown to polarize T cells mediated suppress, often decreasing Th1 responses and augmenting Th2 responses (22-25). The decrease in the Th1 response recalled what we had seen in mice bearing late CMS5 tumors that lost the ability to make CTL against flu virus, a response that requires help by Th1 cells (11). To test if there was a switch in the dominance of Th1 versus Th2 responses as tumor growth progressed, mice were injected with 0.5×106 CMS5 cells. Early and late tumor-bearing mice were primed in vivo with the PR8 flu virus, a Th1 antigen, or with KLH, a Th2 antigen, as described in the Materials and Methods section. After 7 days spleen cells were isolated, restimulated in vitro for 72 hours with APC infected with flu or fed KLH for 84 hours. T cells from early and late-tumor bearing mice stimulated with flu were tested for IFN secretion and those stimulated with KLH were tested for IL-4 and IL-10 secretion in ELISA. The results revealed that T cells from early tumor-bearing mice primed with PR8 made good IFN responses, but those from late tumor-bearing mice were reduced. We showed that Lewis X-containing glycans are expressed on solid tumor cells. Cells were established in culture and stained with 100 ng of unlabeled E.5 monoclonal antibody and a secondary goat anti-mouse IgM-FITC after 24 and 48 hours. Analysis was by performed by flow cytometry.

In contrast, T cells from early tumor-bearing mice primed with KLH secreted very low levels of IL-4 and IL-10, but levels were increased in late tumor-bearing mice. The results are representative of those obtained in 3 experiments. Thus, similar to what is seen in mice with schistosomiasis, Th1 responses decreased, while Th2 responses increased with time as tumors increased in size.

CMS5 Tumors do not Grow in Mice Depleted for CD4+/CD25+ Treg

The presence of CD4+/CD25+Treg cells has been shown to interfere with the rejection of immunogenic tumors (4,5). Moreover, several reports have implicated CD4+/CD25+ cells in fostering the Th2 response that develops to parasites (26). IL-10-dependent and IL-10-independent mechanisms have been implicated in clearance of helminthes and Leishmania by Treg (26,31,32). Since IL-10 secretion by T cells was increased in late tumor-bearing compared to early tumor-bearing mice, and since Th2 responses are favored in late tumor-bearing mice, we tested whether Treg played a role in the growth of CMS5. Experimental mice were depleted of CD4+/CD25+ cells by i.v. injection of 400 ug of anti-CD25 monoclonal antibody (PC61.5.3, rat anti-mouse IgG1, ATCC) on days −5 and −2 prior to the inoculation of CMS5 cells. Control mice received saline. Tumor size in CD25-depleted and control mice was followed with time. The results demonstrated that tumors failed to grow in mice depleted for Treg for up to 30 days, as long as the mice were studied. We showed that tumors do not grow in mice depleted for CD4+/CD25+Treg. Mice were inoculated on day −5 and −2 with 400 ug of anti-CD25 (PC61.5.3, a mouse anti-rat IgG1 Ab, ATCC) i.v., or HBSS as a control, prior to receiving 0.5×106 CMS5 cells s.c. Tumor growth was followed with time. Similar results were seen in 2 experiments. Thus, it appears that the early immunity that develops to CMS5 is sustained in the absence of Treg.

Discussion: Although the downregulation of immunity to pathogens and tumors is a natural event that prevents the development of chronic inflammation as antigen levels decrease, it should occur only after the threat to the host has been dealt with. Premature loss of immunity can be inappropriate and dangerous. In some tumor models, including ours, immunity develops transiently, then is lost. CTL from mice injected 9-10 days earlier with tumor cells lyse CMS5 cells in an antigenically specific fashion. However, tumors continue to increase in size and by day 28 cytotoxicity is no longer detectable (12). As mice lose anti-tumor immunity, they develop massive splenomegaly due to the infiltration of Gr1+/CD11b+ cells (5). In mice immunization with Lewis X-containing glycans, similar to those expressed on schistosome eggs, induces a Th2 response (21). The switch is regulated, at least in part, by the induction of Gr1+/CD11b+ cells. Moreover, evidence suggests that IL-10-secreting CD4+/CD25+Treg contribute to the suppression of Th1 responses and favor the development of Th2 responses during schistosomiasis (26).

In this report, we demonstrate that CMS5 cells express Lewis X-containing glycans also found on schistosome eggs. We also show that as the tumor progresses mice make weaker Th1 responses, while Th2 responses are augmented. Finally, we demonstrate that there is a role for CD4+/CD25+ cells in tumor progression, since tumors are unable to grow in animals depleted of CD4+/CD25+ cells. Taken together, these findings suggest the hypothesis that as the CMS5 tumor increases in size, Lewis X-containing glycans induce Gr1+/CD 11b+ and Treg cells that switch the protective Th1 response to a deleterious Th2 response.

Gr1+/CD11b+ cells, known to suppress T cells, appear to be central to the loss of immunity. First, they increase as immunity to CMS5 wanes and, when levels normalize following tumor resection, anti-tumor immunity returns (5). In mice with anti-viral immunity elimination of Gr1+/CD11b+ cells also is followed by the restoration of T cell mediated cytotoxicity (13). A variety of mechanisms exist by which Gr1+/CD11b+ cells inhibit primed T cells, including the induction of apoptosis or anergy in T cells, the secretion of a variety of soluble mediators, e.g., reactive oxygen species including hydrogen peroxide and nitric oxide, TGF, and arginase 1, leading to direct toxicity, and interference with the Jak3/STAT5 signaling pathways (32;33). Significantly, similar cells have been described in patients, suggesting that these findings may have clinical relevance (29;34-40).

Recent work on the regulation of T cell polarization to pathogens has shown that glycans, including oligosaccharides and lipopolysaccharides expressed by pathogens, play an important role (22-25). A variety of Lewis X-containing N- and O-linked glycoconjugates containing core α3-fucose epitopes from schistosomes have been defined that may influence immune responses (22). Schistosomes and other parasites express glycans that modulate immune responses to favor the production of Th2 cytokines (23,24). Van der Kleij has identified a schistosome-specific phosphatidylserine that activated TLR2, resulting in the generation of Treg that secreted IL-10 (72) We reported that i.p. injection of a dextran conjugate of glycans containing the Lewis X trisaccharide induces Gr1+/CD11b+ cells in the peritoneum within 20 hours (19). The monoclonal E.5 detects Lewis X-containing glycans expressed by schistosome eggs and, using it, we have detected binding on CMS5, MCA38, Lewis Lung, B16 melanoma (FIG. 13A-13D) and CMS4 and CT-26 (results not shown) tumor cell lines. Interestingly, Ghosh et al. reported that tumor-bearing mice lost Th1 responses and maintained Th2 responses (6).

It is striking that certain aspects of the immune response to parasites, schistosomes in particular, resemble that of the immune response to CMS5. First, in both cases, the early Th1 response that develops is followed by a Th2 response. Second, in both cases levels of Gr1+/CD11b+ cells are elevated. Third, both schistosome eggs and CMS5 express Lewis X-containing glycans. Finally, there is a role for CD4+/CD25+ Treg cells that secrete IL-10 and suppress Th1 responses, reducing damage to the liver in schistosomiasis (26) and, based upon our data, preventing rejection of CMS5 cells. Taken together, the results that the mechanism responsible for downregulating Th1 responses and inducing Th2 responses in schistosomiasis may have a broader biological implication and, in the setting of cancer, be deleterious.

### REFERENCES

1. Boon, T. and P. van der Bruggen. 1996. J. Exp. Med. 183:725-729.
2. Boon, T., et al., 1994. Annu. Rev. Immun. 12:337-365.
3. Allison, J. P. 1994. Curr. Opin. Immunol. 6:414-419.
4. Shimizu, J., Yamazaki, S., and Sakaguchi, S. 1999. J. Immunol 163: 5211-5218.
5. Tanaka, H., et al. 2002. J. Immunother. 25: 207-217.
6. Ghosh P, et al., 1995. J. Natl. Cancer Inst. 87:1478-83.
7. Watson, A. G. and D. M. Lopez. 1995. J. Immunol. 155:3124-3134.
8. Fu, E. J., et al., J. Immunother. 20:111-22, 2997.
9. Gansbacher, B., et al., 1990. J. Exp. Med. 172:1217-1224.
10. Salvadori, S., et al., 1994. J. Immunol. 153:5176-5182.
11. Salvadori, S. and K. Zier. 1996. J. Immunol. 156:2927-2932.
12. Salvadori, S., G. Martinelli, and K. Zier. 2000 J. Immunol 164:2214-2220.
13. Young, M. R. I., et al., 1996. J. Immunol. 156:1916-1922.
14. Bronte, V., et al., 1998, J. Immunol 161:5313-5320.
15. Gabrilovich, D. I., et al., 2001. J. Immunol. 166:5398-5406.
16. Mazzoni, A., et al., 2002, J. Immunol. 168:689-695.
17. Rodriguez, P. C., et al., 2004, Cancer Res. 64:5839-5849.
18. Falton, P., Immunopathology of schistosomiasis: a cautionary tale of mice and men. 2000, Immunol. Today. 21:29-35.
19. Atochina, O., et al., 2001. J. Immunol. 167:4293-4302.
20. Terrazas, L. I., et al., 2001, J. Immunol. 167:5294-5303.
21. Okano, M., et al., 2001, J. Immunol. 167:442-450.
22. Nyame, K., Cummings, R. D., R. T. Damian. 1988, J. Parasitol. 74:562-72.
23. Okano, M., et al., 1999, J. Immunol. 163:6712-6717.
24. de Jong, E. C., et al., 2002, J. Immunol. 168:1704-1709.
25. van der Kleij, D., et al., 2003. J. Biol. Chem. 277:48122-48129.
26. McKee, A. S. and Pearce, E. J. 2004. J. Immunol. 173:1224-31.
27. Ko A. I., Drager U. C., D. A. Ham. 1990, Proc. Natl. Acad. Sci. 87:4159-4163.
28. Lopez, C. B., et al., 2001, J Interferon Cytokine Res. 9:763-73.
29. Fuster, M. and Esko, J. D. 2005, Nat Rev Cancer. 5:526-42.
30. Patton, E. A., et al., 2002. Infect. Immun. 70:177-84.
31. Belkaid, Y., et al., 2002. Nature. 420:502-7.
32. Taylor, M. D., et al., 2005, J. Immunol. 174:4924-33.
33. Almand, B et al., 2000, Clin. Cancer Res. 6:1755-1766.
34. Young, M. R. I. and D. M. R. Lathers. 1999, Intl. J. Immunopharm. 21:241-252.

The references cited herein and throughout the specification and examples are herein incorporated by reference in their entirety.

1. A method of treating a disease or disorder in a mammal, comprising administering an effective amount of at least one molecule that will bind to a carbohydrate to the mammal affected with said disease or disorder, wherein the carbohydrate is expressed on the surface of a virus that infects a cell in the mammal with said disease or disorder or said carbohydrate is expressed on the surface of a cell in the mammal affected with said disease or disorder.

2. The method of claim 1, wherein the molecule binds to the glycan epitope LNFPIII.

3. The method of claim 1, wherein the carbohydrate comprises Lewis X trisaccharide.

4. The method of claim 1, wherein the molecule is a monoclonal antibody.

5. The method of claim 4, wherein the antibody is a single chain antibody.

6. The method of claim 5, wherein the single chain antibody is a humanized antibody.

7. The method of claim 4, wherein the antibody is an antibody fragment selected from the group consisting of Fab, F(v), Fab′ and F(ab)2 fragment.

8. The method of claim 1, wherein the disease or disorder is an infectious disease.

9. The method of claim 8, wherein the infectious disease is a primate lentiviral infection.

10. The method of claim 9, wherein the lentiviral infection is an HIV-1 infection.

11. The method of claim 1, wherein the binding of the molecule to the carbohydrate blocks the binding of the virus to its target cell.

12. The method of claim 1, wherein the disease or disorder is a malignancy.

13. The method of claim 12, wherein the malignancy is a solid tumor.

14. The method of claim 13, wherein the solid tumor is colon cancer, melanoma, or lung cancer.

15-29. (canceled)

30. The method of claim 1, wherein the molecule is an antibody.

31. The method of claim 8, wherein the molecule is a humanized single chain antibody that binds to the glycan epitope LNFPIII.

32. The method of claim 13, wherein the molecule is a humanized single chain antibody that binds to the glycan epitope LNFPIII.

33. The method of claim 12, wherein in the molecule is a single chain antibody that binds to the glycan epitope LNFPIII.

34. The method of claim 33, wherein the molecule is a humanized single chain antibody.

35. The method of claim 14, wherein the molecule is a humanized single chain antibody that binds to the glycan epitope LNFPIII.


SEQUENCE LISTING (4 sequences)

SEQ ID NO: 1 - protein, 15 aa, Human immunodeficiency virus type 1
RIQRGPGRAFVTIGK

SEQ ID NO: 2 - DNA, 47 bp, Artificial Sequence
  Description of Artificial Sequence Synthetic primer
ggccatggggcatcatcatcatcatcataaagattccgcgggagaag

SEQ ID NO: 3 - DNA, 29 bp, Artificial Sequence
  Description of Artificial Sequence Synthetic primer
ggctcgagatcatttgtccatccttcatc

SEQ ID NO: 4 - protein, 6 aa, Artificial Sequence
  Description of Artificial Sequence Synthetic 6x His tag
HHHHHH